(19) United States
(12) Patent Application Publication
(10) Pub. No.: US 2026/0026740 A1
(43) Pub. Date: Jan. 29, 2026

(54) POSTURE MEASUREMENT APPARATUS, POSTURE MEASUREMENT METHOD, AND NON-TRANSITORY COMPUTER READABLE MEDIUM

(71) Applicants: NEC Corporation, Tokyo (JP); INSTITUTE OF SCIENCE TOKYO, Tokyo (JP)

(72) Inventors: Shuhei NOYORI, Tokyo (JP); Keisuke SUZUKI, Tokyo (JP); Yuki KOSAKA, Tokyo (JP); Akimoto NIMURA, Tokyo (JP); Koji FUJITA, Tokyo (JP); Takuya IBARA, Tokyo (JP)

(73) Assignees: NEC Corporation, Tokyo (JP); INSTITUTE OF SCIENCE TOKYO, Tokyo (JP)

(21) Appl. No.: 19/276,307

(22) Filed: Jul. 22, 2025

(30) Foreign Application Priority Data
Jul. 29, 2024 (JP) 2024-121730

(51) Int. Cl. A61B 5/00; A61B 5/107

(52) U.S. Cl. CPC A61B 5/4561; A61B 5/1071; A61B 5/1079

(57) ABSTRACT

A posture measurement apparatus includes at least one memory configured to store instructions, and at least one processor configured to execute the instructions to hold a shape model representing a shape of a back of a subject as a function of a plurality of parameters, execute calculation processing of calculating a value of a predetermined objective function using a candidate point cloud as a candidate of a point cloud indicating the shape of the back of the subject and the shape model as inputs, and execute an optimization processing of correcting the parameters in such a way as to minimize the value of the predetermined objective function using a constraint condition of the parameters and the shape model as inputs.

### INCORPORATION BY REFERENCE

This application is based upon and claims the benefit of priority from Japanese patent application No. 2024-121730, filed on Jul. 29, 2024, the disclosure of which is incorporated herein in its entirety by reference.

### TECHNICAL FIELD

The present disclosure relates to a posture measurement apparatus, a posture measurement method, a program, and a posture measurement system.

### BACKGROUND ART

In recent years, with an increase of self-care need and online training for symptoms of musculoskeletal system such as low back pain, there is an increasing need to accurately and quantitatively evaluate posture using an image obtained by a camera provided in a terminal device such as a smartphone.

Patent Literature 1 discloses a posture evaluation apparatus that includes a spinal column extracting means, a feature calculating means, and a state estimating means and can evaluate a posture relatively inexpensively with high accuracy. The spinal column extracting means extracts a spinal column edge point cloud including a predetermined number of points representing a spinal column shape on an image based on the image obtained by capturing an image of a side surface of a body of a subject and position information about at least a cervical vertebra, a hip joint, and a knee joint of the body on the image. The feature calculating means calculates at least a feature related to the spinal column based on the position information and the spinal column edge point cloud. The state estimating means estimates at least a state of the spinal column based on the calculated feature.
Patent Literature 1: International Patent Publication No. WO 2023/188796

### SUMMARY

In the case of evaluating the posture using the image, a shape of a back surface of the body is hidden by the clothing in a case where an evaluation subject wears thick clothing or bends backward. Therefore, the shape of the back that appears if the same posture is taken in a state where no clothing is worn cannot be accurately estimated from the image, whereby the posture cannot be accurately evaluated. Patent Literature 1 cannot solve such a problem.

Therefore, it is desired to develop a technique for simply and accurately measuring the shape of the back of the subject even in a scene where the shape of the back of the subject cannot be accurately grasped due to the presence of the clothing of the subject.

An example object of the present disclosure is to provide a posture measurement apparatus, a posture measurement method, a program, and a posture measurement system capable of simply and accurately measuring a shape of a back of a subject even in a scene where the shape of the back of the subject cannot be accurately grasped due to presence of clothes of the subject.

In a first example aspect of the present disclosure, a posture measurement apparatus includes at least one memory configured to store instructions, and at least one processor configured to execute the instructions to: hold a shape model representing a shape of a back of a subject as a function of a plurality of parameters, execute calculation processing of calculating a value of a predetermined objective function using a candidate point cloud as a candidate of a point cloud indicating the shape of the back of the subject and the shape model as inputs, and execute optimization processing of correcting the parameters in such a way as to minimize the value of the predetermined objective function using a constraint condition of the parameters and the shape model as inputs.

In a second example aspect of the present disclosure, in a posture measurement method, a posture measurement apparatus holds a shape model representing a shape of a back of a subject as a function of a plurality of parameters, calculates a value of a predetermined objective function using a candidate point cloud as a candidate of a point cloud indicating the shape of the back of the subject and the shape model as inputs, and corrects the parameters in such a way as to minimize the value of the predetermined objective function using a constraint condition of the parameters and the shape model as inputs.

In a third example aspect of the present disclosure, a program causes a computer to execute processing of holding a shape model representing a shape of a back of a subject as a function of a plurality of parameters, calculating a value of a predetermined objective function using a candidate point cloud as a candidate of a point cloud indicating the shape of the back of the subject and the shape model as inputs, and correcting the parameters in such a way as to minimize the value of the predetermined objective function using a constraint condition of the parameters and the shape model as inputs.

An example advantage according to the present disclosure is to be able to provide a posture measurement apparatus, a posture measurement method, a program, and a posture measurement system capable of simply and accurately measuring a shape of a back of a subject even in a scene where the shape of the back of the subject cannot be accurately grasped due to presence of clothes of the subject.

### BRIEF DESCRIPTION OF DRAWINGS

The above and other aspects, features and advantages of the present disclosure will become more apparent from the following description of certain exemplary embodiments when taken in conjunction with the accompanying drawings, in which:

FIG. 1 is a block diagram illustrating a configuration example of a posture measurement apparatus according to the present disclosure;

FIG. 2 is a block diagram illustrating a configuration example of a posture measurement apparatus according to the present disclosure;

FIG. 3 is a view illustrating an example of a back shape model held for an image acquired by the posture measurement apparatus according to the present disclosure;

FIG. 4 is a view illustrating another example of the back shape model held for the image acquired by the posture measurement apparatus according to the present disclosure;

FIG. 5 is a schematic diagram illustrating an example of a calculation method in the back shape model of FIG. 3 and a calculation method of an objective function;

FIG. 6 is a diagram illustrating a mathematical expression illustrating a processing example in the posture measurement apparatus according to the present disclosure;

FIG. 7 is a schematic diagram illustrating an example of candidate point cloud extraction processing in the posture measurement apparatus according to the present disclosure;

FIG. 8 is a schematic diagram illustrating another example of candidate point cloud extraction processing in the posture measurement apparatus according to the present disclosure;

FIG. 9 is a schematic diagram illustrating an example of end point determination processing in the posture measurement apparatus according to the present disclosure;

FIG. 10 is a schematic diagram illustrating an example of edge addition processing in the posture measurement apparatus according to the present disclosure;

FIG. 11 is a schematic diagram illustrating an example of body keypoint extraction processing in the posture measurement apparatus according to the present disclosure;

FIG. 12 is a schematic diagram illustrating an example of body keypoints extracted in body keypoint extraction processing in the posture measurement apparatus according to the present disclosure;

FIG. 13 is a view illustrating an example of an image displayed on a display unit in the posture measurement apparatus according to the present disclosure;

FIG. 14 is a schematic diagram illustrating an example of imaging angle determination processing in the posture measurement apparatus according to the present disclosure;

FIG. 15 is a schematic diagram illustrating an example of imaging angle determination processing in the posture measurement apparatus according to the present disclosure;

FIG. 16 is a flowchart illustrating a posture evaluation method according to the present disclosure;

FIG. 17 is a diagram illustrating an estimation result of a shape of a back in a forward bending posture according to a comparative example;

FIG. 18 is a diagram illustrating an estimation result of a shape of a back in a backward bending posture according to a comparative example; and

FIG. 19 is a diagram illustrating a configuration example of a posture evaluation system according to the present disclosure.

### EXAMPLE EMBODIMENT

Hereinafter, example embodiments will be described with reference to the drawings. To clarify description, in the following description and drawings, omission and simplification are made as appropriate. In each drawing, the same elements are denoted by the same reference signs, and redundant description will be omitted as necessary.

### First Example Embodiment

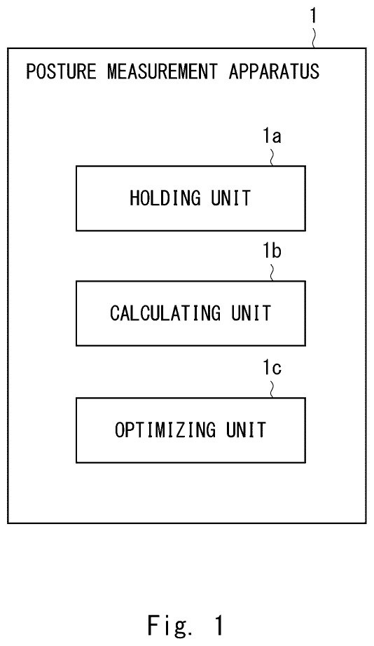

A configuration example of a posture measurement apparatus 1 will be described hereinafter with reference to FIG. 1.

As illustrated in FIG. 1, the posture measurement apparatus 1 includes a holding unit 1a, a calculating unit 1b, and an optimizing unit 1c.

The holding unit 1a holds a shape model representing a shape of a back of a subject as a function of a plurality of parameters. The holding unit 1a can also be referred to as a shape model holding unit. Since this shape model is a shape model of the back, the model is hereinafter referred to as a back shape model. Since the back shape model represents the shape of the back, the back shape model represents the shape of a spinal column in a pseudo manner.

The calculating unit 1b calculates a value of a predetermined objective function using a candidate point cloud as a candidate of a point cloud indicating the shape of the back of the subject and the back shape model as inputs. The calculating unit 1b can also be referred to as an objective function calculating unit.

The optimizing unit 1c corrects the plurality of parameters in such a way as to minimize a value of the predetermined objective function using constraint conditions of the plurality of parameters and the back shape model as inputs. The optimizing unit 1c can be referred to as a parameter optimizing unit.

The posture measurement apparatus 1 can use the optimized back shape model as a shape model representing the shape of the back of the subject, thereby measuring the shape of the back. Since the posture measurement apparatus 1 is a device that estimates the shape of the back by optimization, the device can also be referred to as a posture estimation device.

The back shape model may be a shape model that represents the shape of the back of the subject in a sagittal plane as a function of a plurality of parameters. In this case, the input candidate point cloud can be a candidate of a point cloud indicating the shape of the back of the subject in the sagittal plane. However, the back shape model and the candidate point cloud are not limited to within the sagittal plane, and may be within a plane having an angle with respect to the sagittal plane as long as the shape of the back can be grasped.

As described above, the posture measurement apparatus 1 can execute the posture measurement method. In this posture measurement method, the back shape model is held, the value of the predetermined objective function is calculated using the candidate point cloud and the back shape model as inputs, and the plurality of parameters is corrected in such a way as to minimize the value of the predetermined objective function using the constraint conditions of the plurality of parameters and the back shape model as inputs. In addition, this posture measurement method can also be achieved by a program and hardware that executes the program. This program refers to a program that causes a computer to execute the processing described in the posture measurement method described above.

Although not illustrated, the posture measurement system can be configured by including a posture measurement apparatus 1 and a terminal device capable of communicating with the posture measurement apparatus 1. In this case, the posture measurement apparatus 1 may include, in addition to the holding unit 1a, the calculating unit 1b, and the optimizing unit 1c, an acquiring unit that acquires a candidate point cloud to be a candidate of a point cloud indicating the shape of the back of the subject or an image to be an extraction source of the candidate point cloud from a terminal device.

According to the present example embodiment, since the held back shape model is optimized based on the input candidate point cloud, the shape of the back of the subject can be simply and accurately measured even in a scene where the shape of the back of the subject cannot be accurately grasped due to the presence of clothes of the subject.

### Second Example Embodiment

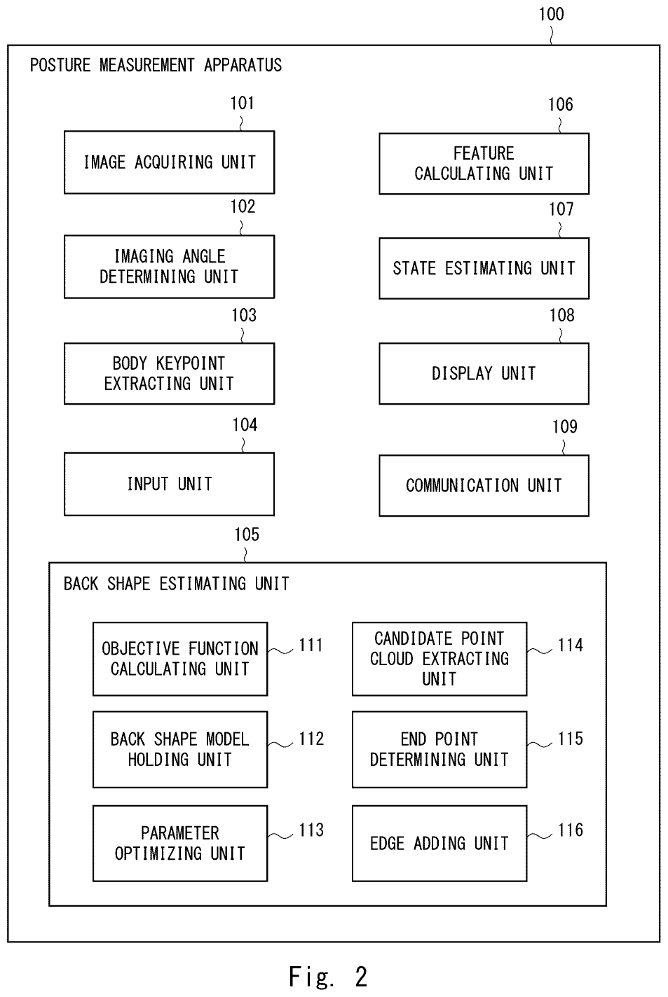

A configuration example of a posture measurement apparatus 100 will be described with reference to FIG. 2. The posture measurement apparatus 100 includes the posture measurement apparatus 1 illustrated in FIG. 1. The posture measurement apparatus 100 includes a back shape model holding unit 112 as an example of the holding unit 1a, an objective function calculating unit 111 as an example of the calculating unit 1b, and a parameter optimizing unit 113 as an example of the optimizing unit 1c.

The posture measurement apparatus 100 illustrated in FIG. 2 is, for example, a user terminal such as a smartphone, a tablet terminal, or a personal computer possessed by the user. The user includes both a subject whose posture is to be measured by the posture measurement apparatus 100 and a measurer who measures the posture of another person using the posture measurement apparatus 100. In a case where the subject measures his/her own posture using the posture measurement apparatus 100 in self-training or the like, the subject is also a measurer. Furthermore, in a case where the measurer measures the posture of another person using the posture measurement apparatus 100, the measurer is, for example, a therapist or a trainer.

As illustrated in FIG. 2, the posture measurement apparatus 100 includes an image acquiring unit 101, an imaging angle determining unit 102, a body keypoint extracting unit 103, an input unit 104, a back shape estimating unit 105, a feature calculating unit 106, a state estimating unit 107, a display unit 108, and a communication unit 109. The input unit 104 and the display unit 108 may have one configuration as a touch-panel-equipped display, or may be provided separately. The back shape estimating unit 105 can include a candidate point cloud extracting unit 114, an end point determining unit 115, and an edge adding unit 116 in addition to the objective function calculating unit 111, the back shape model holding unit 112, and the parameter optimizing unit 113. In addition, the posture measurement apparatus 100 can include a storage unit (not illustrated). The storage unit stores, for example, the back shape model held by the back shape model holding unit 112.

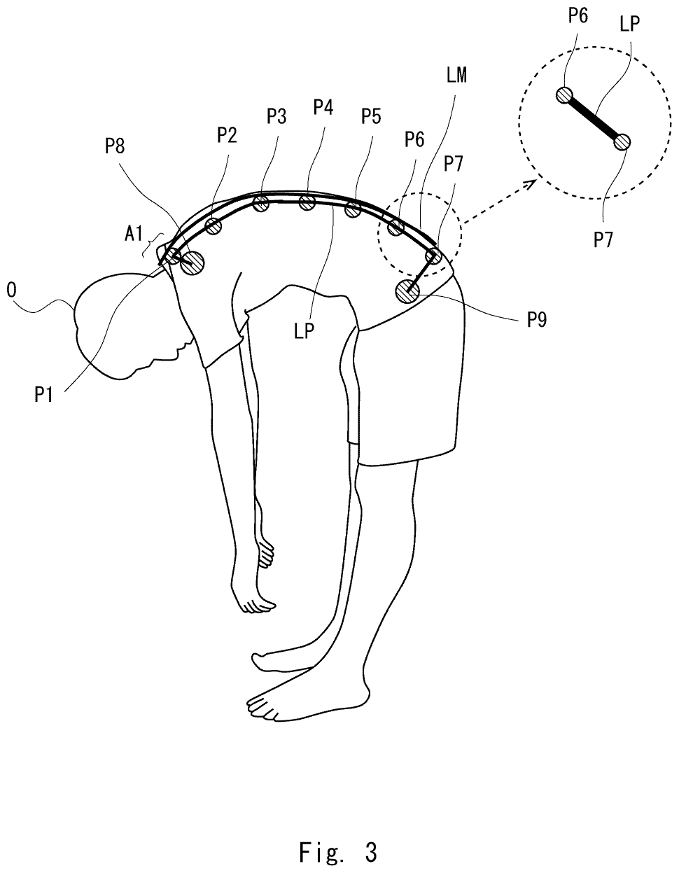

The image acquiring unit 101 acquires an image obtained by capturing an image of the side surface of the body of the subject for posture measurement. The image acquiring unit 101 may include an image capturing apparatus that captures an image. An example of an image obtained by capturing an image of the side surface of the body of the subject by the image acquiring unit 101 is illustrated in FIG. 3. In the image illustrated in FIG. 3, the side surface of a person O associated to the subject bending forward is reflected. Here, the image acquired by the image acquiring unit 101 is a two-dimensional image, and may be a two-dimensional RGB image. The image acquiring unit 101 outputs the acquired image to the body keypoint extracting unit 103, the back shape estimating unit 105, or both of them. This output destination differs depending on the processing example adopted in the present example embodiment.

The image acquiring unit 101 may acquire a moving image obtained by capturing an image of a side surface of the body of the subject of posture measurement. In this case, the user may operate the input unit 104 to designate a time point at which the posture is measured or evaluated. Then, the image acquiring unit 101 may output the image at the time point designated by the user to an output destination.

The input unit 104 receives an operation instruction from the user. The input unit 104 may be configured by a keyboard, or may configure a display with a touch panel in combination with the display unit 108 as described above. The input unit 104 may include a keyboard or a touch panel connected to a main body of the posture measurement apparatus 100.

The display unit 108 displays images such as captured and acquired images and measurement results. The display unit 108 includes various display means such as a liquid crystal display (LCD) and a light emitting diode (LED).

The communication unit 109 communicates with an external server, another terminal device, or the like. The communication unit 109 may include an antenna (not illustrated) that performs wireless communication, or may include an interface such as a network interface card (NIC) for performing wired communication. The image acquiring unit 101 may be configured to acquire an image from the image capturing apparatus via the communication unit 109. The input unit 104 may receive an operation instruction by the user from a keyboard or a touch panel connected to the main body of the posture measurement apparatus 100 via the communication unit 109.

First, as a main configuration in the present example embodiment, the objective function calculating unit 111, the back shape model holding unit 112, and the parameter optimizing unit 113 in the back shape estimating unit 105 will be described.

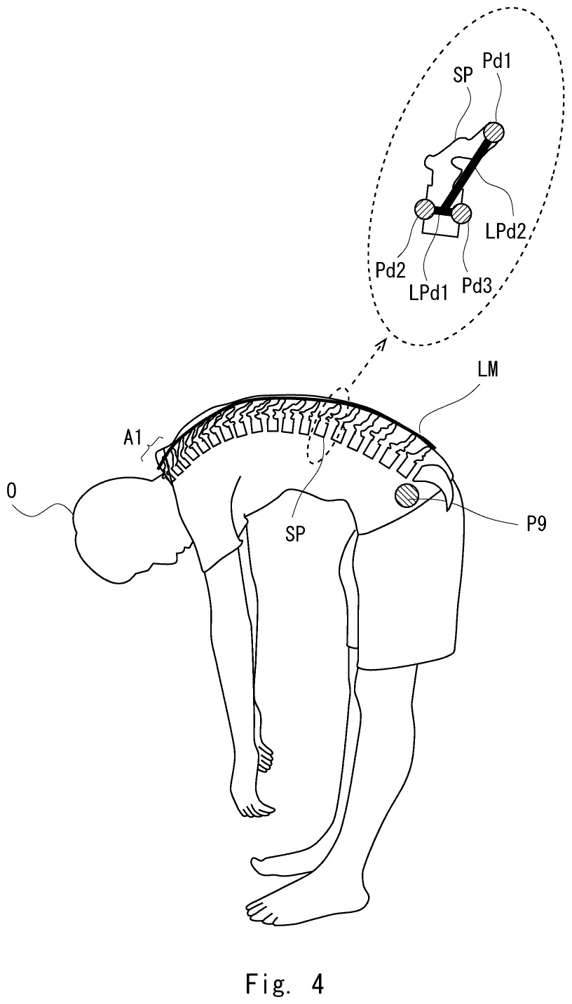

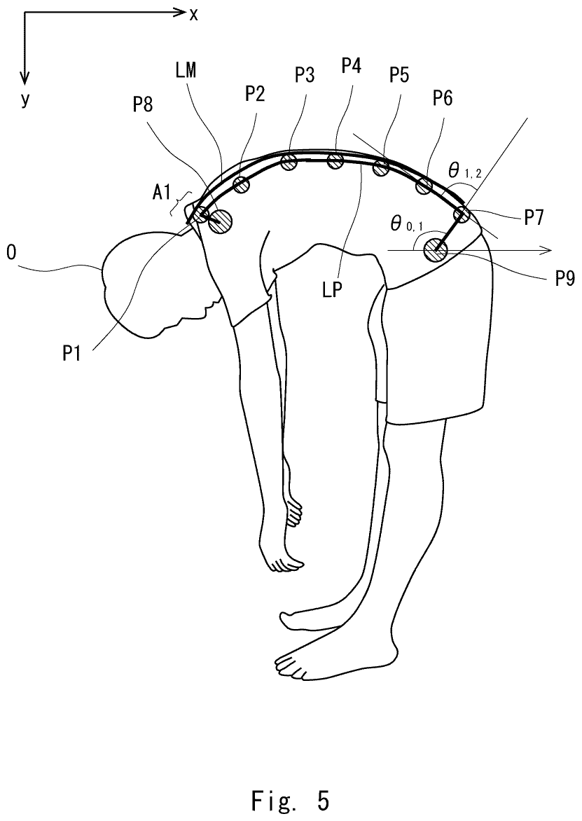

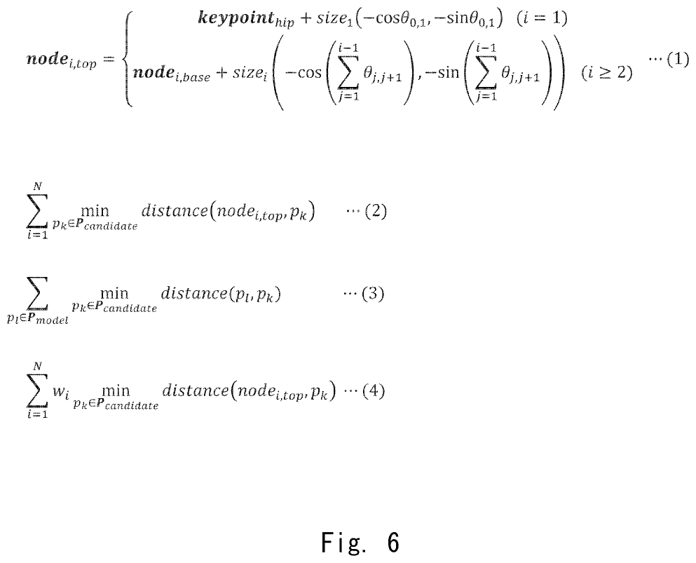

Hereinafter, the back shape model held by the back shape model holding unit 112 and the optimization thereof in the present example embodiment will be exemplified with reference to FIGS. 3 to 6. FIG. 3 is a view illustrating an example of a back shape model held for an image acquired by the posture measurement apparatus 100 according to the present disclosure. FIG. 4 is a view illustrating another example of the back shape model held for the image acquired by the posture measurement apparatus 100 according to the present disclosure. FIG. 5 is a schematic diagram illustrating an example of a calculation method in the back shape model of FIG. 3 and a calculation method of an objective function. FIG. 6 is a diagram illustrating a mathematical expression illustrating a processing example in the posture measurement apparatus according to the present disclosure.

The back shape model will be described on the assumption that the back shape model is a model representing the shape of the back of the subject in the sagittal plane, but is not limited thereto as described in the first example embodiment. The back shape model can be a model in which the shape of the spinal column of the subject is expressed by a plurality of parameters as the shape of the back of the subject. In this case, the back shape estimating unit 105 can also be referred to as a spinal column shape estimating unit, a spinal column extracting unit, or the like.

The back shape model held by the back shape model holding unit 112 receives, as input, for example, the size of each part, the angle of each part in the sagittal plane, and the position information about the neck and the hip joint orthogonally projected on the sagittal plane of the subject, and reconfigures itself based on the received information. The back shape model reconfigured in this manner is updated as an output with respect to the input, and is held in the back shape model holding unit 112.

As described above, the plurality of parameters in the back shape model can include, for example, at least the size of each part configuring the back shape model, the angle representing the posture in the sagittal plane, and the position information indicating the positions of the neck and the hip joint in the sagittal plane. Each part configuring the back shape model may include at least a part of the part of the spinal column.

The angle representing the posture in the sagittal plane can be an angle of each part configuring the back shape model or an angle of some parts configuring the back shape model. The angle of the part may be an angle with respect to a reference plane such as a horizontal plane or a vertical plane, or may be a relative angle with respect to an adjacent part. In addition, the angle representing the posture in the sagittal plane may be, for example, an angle or a relative angle with respect to the reference plane of a connecting portion between adjacent parts or a line segment connecting between each part and a predetermined pair of parts. Furthermore, the position information can be information indicating the positions of the neck and the hip joint orthogonally projected on the sagittal plane of the subject.

As illustrated in FIG. 3, the back shape model may be a model expressed by a smaller number of parts than the number of vertebrae. This model is a model simplified in such a way as to be a simple model, and can be referred to as a simple model.

The exemplified simplified model is configured by parts associated to a pelvic key point P7, a lumbar vertebra lower half key point P6, a lumbar vertebra upper half key point P5, a lower thoracic vertebra lower half key point P4, a lower thoracic vertebra upper half key point P3, an upper thoracic vertebra lower half key point P2, and an upper thoracic vertebra upper half key point P1 starting from a hip joint key point P9. The key point P1 can be, for example, a key point associated to the upper end of the upper thoracic vertebrae. Further, the illustrated simplified model may further include a part connecting the key point P1 at the upper end of the upper thoracic vertebrae and the key point P8 at the base of the neck. In FIG. 3, as illustrated by enlarging the key points P6 and P7 and the parts between the key points P6 and P7, in the exemplified simplified model, a connecting portion between the parts may be defined as a part LP for each of the adjacent parts.

In the simplified model, a shape LM of the back of the subject O can be expressed by each of these parts. Alternatively, as illustrated as the shape LM of the back, each of the key points P1 to P7 can be expressed as a part of a point cloud moved to the outside of the body by a predetermined distance with respect to each of the key points P1 to P7 and a part of a connecting portion of the point cloud.

Furthermore, the back shape model can be a detailed model that is a more detailed model than the above-described simple model. The detailed model may be a model close to the shape of the actual vertebra, or may be a simplified model that simulates only the vertebra and the spinous process portion as illustrated in FIG. 4. FIG. 4 depicts a small number of vertebrae for convenience. The detailed model illustrated in FIG. 4 is a model in which the shape LM of the back of the subject O is represented by a part having the same number of vertebrae as the actual number or a slightly reduced number and having a portion similar to the spinous process for each vertebrae.

Specifically, as illustrated in an enlarged manner in FIG. 4, each vertebra SP can be expressed as follows, for example. That is, each vertebra SP can be expressed by key points Pd1, Pd2, and Pd3 representing the target vertebra SP, a part LPd1 of a connecting portion between the key points Pd2 and Pd3, and a part LPd2 connecting a midpoint of the part LPd1 and the key point Pd1. The parts Pd1 and LPd2 are parts representing the spinous process. The detailed model illustrated in FIG. 4 is a model including a part associated to the key point P9 of the hip joint in addition to these. This detailed model can be configured by, for example, parts associated to a sacrum part, lumbar vertebrae 1 to 5, thoracic vertebrae 1 to 12, and a cervical vertebra 7 of the pelvis starting from the key point P9 of the hip joint, but the vertebrae included in the configuration is not limited to this example.

However, since it is extremely difficult to accurately reproduce the shape of the vertebra of the human without using a see-through imaging method such as computed tomography (CT) or magnetic resonance imaging (MRI), it can be said that it is difficult to perform modeling with high accuracy even if the detailed model as illustrated in FIG. 4 is adopted. Therefore, in consideration of the calculation amount, it can be said that the simplified model illustrated in FIG. 3 is better than the detailed model illustrated in FIG. 4.

A specific calculation method of the back shape model if the simple model illustrated in FIG. 3 is adopted as the back shape model will be described. Consider a case where there are N parts in the simplified model. The parts are part₁, . . . , partᵢ, . . . , part_{N} from the side closer to the hip joint. Further, the hip joint side of both ends of partᵢ is referred to as a base (node_{i,base}) and a tip (node_{i, top}). At this time, the position of the leading end node_{i,top} of partᵢ is obtained by, for example, Expression (1) in FIG. 6.

Bold elements in Expression (1) represent a two-dimensional vector of (x coordinate, y coordinate). In the following description, although not indicated in bold for convenience, the same elements as the elements indicated in bold in Expression (1) similarly represent a two-dimensional vector.

The keypointₕᵢₚ indicates the position of the hip joint. In addition, since the adjacent parts are connected, node_{i,base}=node_{i-1,top}. sizeᵢ refers to the size of partᵢ, specifically a distance between the base and the tip. If the reference part sizes size_{1.0}, . . . , size_{i,0}, . . . , size_{N,0} are given, the ratio r₁, . . . , rᵢ, . . . , r_{N} to the size of the reference part can be used to define sizeᵢ=rᵢsize_{i,0}. Further, θ_{i,i+1} represents an angle formed by partᵢ and partᵢ₊₁. Here, for example, the clockwise direction can be defined as positive with the same direction as partᵢ as 0. As an example, θ_{1,2} can be defined as illustrated in FIG. 5, and other angles can be similarly defined. The angle θ_{0,1} of part₁ can be defined with reference to an angle with respect to the vector from the hip joint to the cervical vertebra, for example, although there is no reference part₀.

The edge is approximated using the position of node_{i,top} obtained by Expression (1). Here, the edge refers to a shape of the back that appears in a case where the same posture is taken in a state where no clothing is worn. Therefore, the candidate point cloud input by the objective function calculating unit 111 to be described later is referred to as an edge candidate point cloud. Hereinafter, the approximated edge point cloud is referred to as P_{model}.

The processing of approximating the edge can be executed by fitting an nth-order function or a spline function to {node_{i,top}}_{i=1, . . . , N}. In a case where the detailed model is used, an n-th order function or a spline function may be fitted to the point sequence of the spinous process associated portion.

In addition, in a case where it is clear that node_{i,top} is not present on the body surface, such as a part connecting the upper end of the upper half of the upper thoracic vertebrae and the base of the neck, the position information may not be used for approximation of the edge. The image size may be normalized before the calculation of the back shape model. In addition, although a method of determining node_{i,top} based on the hip joint is adopted, other parts or key point positions may be used as a reference.

A specific calculation method in the objective function calculating unit 111 will be described. The objective function calculating unit 111 inputs the back shape model and the edge candidate point cloud P_{candidate} in the sagittal plane of the subject. The edge candidate point cloud P_{candidate} may be extracted from the image by a method to be described later. The edge candidate point cloud P_{candidate} is a point cloud that is a candidate of an edge, that is, a candidate of a point cloud of an edge. In other words, the edge candidate point cloud P_{candidate} is a point cloud indicating an edge as viewed, that is, an edge as indicated by the captured image, and is a point cloud including the sagging of the clothing and the like.

Then, based on the input information, the objective function calculating unit 111 calculates a value of a predetermined objective function from P_{candidate} and {node_{i,top}}_{i=1, . . . and N} as a first method, or from P_{candidate} and P_{model} as a second method. P_{model} is an edge point cloud approximated by the back shape model as described above.

In the first method, as exemplified by Expression (2) in FIG. 6, the objective function calculating unit 111 obtains the distance to the closest point in P_{candidate} for each point of {node_{i, top}}_{i=1, . . . , N} and calculates the sum thereof. As described above, the objective function calculating unit 111 may calculate, as the value of the predetermined objective function, the sum of the distances to the closest point in the edge candidate point cloud for each part indicated by the back shape model. That is, the objective function calculating unit 111 may obtain the distance to the closest point in the edge candidate point cloud for each part indicated by the back shape model, and calculate the sum of the obtained distances as the value of the predetermined objective function.

In the second method, as exemplified by Expression (3) in FIG. 6, the objective function calculating unit 111 obtains the distance to the closest point in the P_{candidate} for each point of the P_{model}, and calculates the sum thereof. As described above, the objective function calculating unit 111 may calculate, as the value of the predetermined objective function, the sum of the distances to the closest point in the approximation candidate point cloud for each point of an approximation candidate point cloud obtained by approximating the edge candidate point cloud based on each part indicated by the back shape model. That is, the objective function calculating unit 111 may obtain the distance to the closest point in the edge candidate point cloud for each point of the approximation candidate point cloud obtained by approximating the edge candidate point cloud based on each part indicated by the back shape model, and calculate the sum of the obtained distances as the value of the predetermined objective function.

In addition, in any of the first method and the second method described above, different weights may be given to each node or section in the calculation of the value. That is, for example, in the first method, the objective function calculating unit 111 may calculate, as the value of the predetermined objective function, the weighted sum of the distances to the closest point in the edge candidate point cloud for each part indicated by the back shape model. For example, in the second method, the objective function calculating unit 111 may calculate, as the value of the predetermined objective function, the weighted sum of the distances to the closest point in the candidate point cloud for each point of the approximation candidate point cloud obtained by approximating the candidate point cloud based on each part indicated by the back shape model.

The consideration of the weight in the first method will be specifically described. Expression (2) in FIG. 6 is expressed by Expression (4) in FIG. 6 using weights w₁, . . . , wᵢ, . . . , and w_{N} (0≤wᵢ≤1). For each node or section, how close the back shape model should be to the P_{candidate} is adjusted. A smaller weight allows the back shape model to be farther from the P_{candidate}. In a case where there is a high possibility that the deviation of the P_{candidate} from the original shape of the back is large, the weight is reduced to perform adjustment in such a way that the back shape model is hardly optimized in a wrong direction. Although the description thereof will be omitted, consideration of the weight in the second method can also be processed in a similar way, and it is sufficient that an expression obtained by multiplying each element of the summation target of Expression (3) by wᵢ is added.

The parameter optimizing unit 113 uses the constraint conditions of the parameters and the back shape model as inputs together with, for example, position information about the neck in the sagittal plane of the subject, and corrects the parameters of the back shape model in such a way as to minimize the value of the objective function.

A specific calculation method in the parameter optimizing unit 113 will be described. The position information about the neck of the subject in the sagittal plane, which is one of the inputs, can be given as an operation input from the input unit 104 by the user or can be estimated from the image acquired by the image acquiring unit 101.

The position information is referred to as position information about the “neck” for convenience, but position information about predetermined vertebrae among seven vertebrae configuring the cervical vertebrae can be used. For example, the position information about the ridge vertebrae, which is the cervical vertebra 7, can be used as the position information. Alternatively, position information indicating an intermediate position between the cervical vertebra 7 and the thoracic vertebra 1 may be used as the position information about the neck. Furthermore, information indicating a position near the middle between the cervical vertebra 7 and the sternal pattern, which is a position estimated as a key point of the “neck” in a general body keypoint extraction model, can also be used as the position information about the neck. In this case, since a deviation from the position of the ridge vertebrae occurs, a part connecting the key point of the “neck” and the upper end of the ridge vertebrae or the upper half of the upper thoracic vertebrae is added to the back shape model, and the calculation of the objective function and the parameter optimization are performed, in such a way that the parameter can be accurately estimated.

As a constraint condition of a parameter, which is one of inputs, a predetermined constraint condition is used. As an example of the constraint condition, for example, at least one of the following first to third conditions can be adopted.

A first condition is that an error between the position of the neck and the position of the neck calculated from the back shape model falls within a predetermined error range. That is, the constraint conditions of the plurality of parameters can include a condition that the position information as a part of the plurality of parameters and the candidate position information indicating the positions of the neck and the hip joint in the sagittal plane as the edge candidate point cloud match within a predetermined error range. The range of the predetermined error here can be determined by, for example, a lower limit and an upper limit of the distance between the two positions. Since the distance does not take a negative value, it can be said that it is desirable that the lower limit is 0 and the upper limit is as small as possible.

A second condition is that the size of each part of the back shape model falls within a predetermined range. The predetermined range can be defined by, for example, a lower limit and an upper limit of the size of each part. In addition, the second condition may be a condition that the ratio to the initial value and the ratio between the parts fall within a predetermined range, instead of an absolute value of the size.

A third condition is that the angle of the part falls within a predetermined range. The predetermined range can be defined by, for example, a lower limit and an upper limit of a relative angle between the parts. The angle of the part is associated with a range of motion between vertebrae in the human spinal column. Therefore, within the predetermined range here, the range of motion of bending and extension in the sagittal plane known in various documents may be provided as a lower limit or an upper limit, or practically possible values may be individually set for several postures such as a forward bending posture and a backward bending posture.

The parameter optimizing unit 113 uses such a parameter constraint condition and the back shape model as inputs together with, for example, position information about the neck in the sagittal plane of the subject, and corrects the parameters of the back shape model in such a way as to minimize the value of the objective function. For the correction, for example, an algorithm for solving a constrained optimization problem, for example, sequential quadratic programming, or the like can be used. Since the aforementioned objective function cannot be differentiated analytically, numerical differentiation may be used to obtain the gradient. Then, as a result of the correction, the parameter optimizing unit 113 outputs a parameter of the back shape model that minimizes the objective function.

As described above, the back shape estimating unit 105 inputs the edge candidate point cloud and the position information indicating the positions of the neck and the hip joint in the sagittal plane, and outputs the optimized back shape model as a result of estimating the edge that is the shape of the back appearing if the same posture is taken in a state where the garment is not worn. The input position information can also be referred to as joint position information. The joint position information can include information indicating positions of joints other than the neck and the hip joint.

The objective function calculating unit 111, the back shape model holding unit 112, and the parameter optimizing unit 113, which are main components in the present example embodiment, have been mainly described above. In the present example embodiment, some of the other components illustrated in FIG. 2 are not essential components, and various variations of the configuration of the posture measurement apparatus 100 will be described below.

For example, the posture measurement apparatus 100 can be configured to include at least one of a candidate point cloud extracting unit 114, an end point determining unit 115, and an edge adding unit 116 in the back shape estimating unit 105 in addition to the above main components. A configuration example in which the back shape estimating unit 105 includes the candidate point cloud extracting unit 114 will be described later as variation 1. A configuration example in which the back shape estimating unit 105 includes the end point determining unit 115 will be described later as variation 2. A configuration example in which the back shape estimating unit 105 includes the edge adding unit 116 will be described later as variation 3.

Furthermore, the posture measurement apparatus 100 can also be configured to include at least one of a body keypoint extracting unit 103, an input unit 104, a feature calculating unit 106, and a display unit 108 in addition to the above-described main components. A configuration example in which the posture measurement apparatus 100 includes the body keypoint extracting unit 103 will be described later as variation 4. A configuration example in which the posture measurement apparatus 100 includes the feature calculating unit 106 will be described later as variation 5. Furthermore, in a case where the posture measurement apparatus 100 includes the state estimating unit 107, the posture measurement apparatus includes the feature calculating unit 106. A configuration example in which the posture measurement apparatus 100 includes the feature calculating unit 106 and the state estimating unit 107 will be described later as variation 6.

A configuration example in which the posture measurement apparatus 100 includes the input unit 104 will be described later as variation 7. A configuration example in which the posture measurement apparatus 100 includes the display unit 108 will be described later as variation 8. In addition, in the case of including the image acquiring unit 101, the posture measurement apparatus 100 includes a candidate point cloud extracting unit 114 or a body keypoint extracting unit 103. With the configuration including the body keypoint extracting unit 103, the candidate point cloud extracting unit 114, and the image acquiring unit 101, the posture measurement apparatus 100 can automatically evaluate the posture from the image capturing, that is, can function as a posture evaluation apparatus. A configuration example in which the posture measurement apparatus 100 includes the image acquiring unit 101 will be described later as variation 9. Furthermore, in a case where the posture measurement apparatus 100 includes the imaging angle determining unit 102, the posture measurement apparatus also includes the image acquiring unit 101. A configuration example in which the posture measurement apparatus 100 includes the image acquiring unit 101 and the imaging angle determining unit 102 will be described later as variation 10.

### <Variation 1>

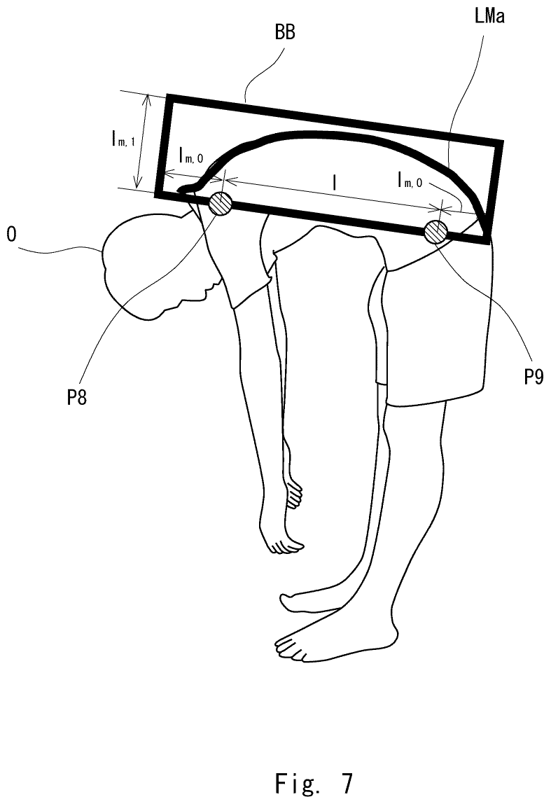

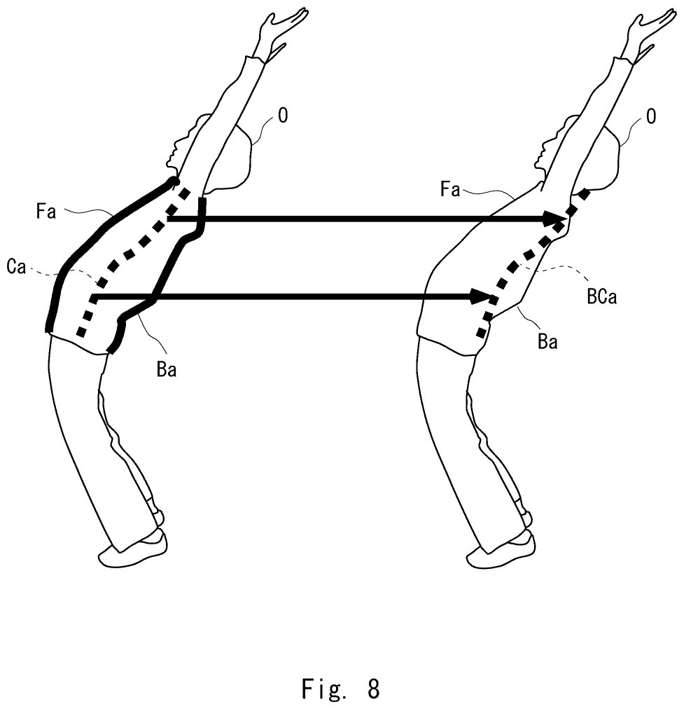

A configuration example in which the back shape estimating unit 105 includes the candidate point cloud extracting unit 114 will be described as variation 1 with reference to FIGS. 7 and 8. FIG. 7 is a schematic diagram illustrating an example of candidate point cloud extraction processing in the posture measurement apparatus according to the present disclosure. FIG. 8 is a schematic diagram illustrating another example of the candidate point cloud extraction processing in the posture measurement apparatus according to the present disclosure.

In variation 1, the back shape estimating unit 105 inputs the image and the joint position information acquired by the image acquiring unit 101, and outputs an optimized back shape model.

An example of such a configuration that is effective particularly in the case of an upright position or a forward bending posture will be described with reference to FIG. 7. First, the candidate point cloud extracting unit 114 inputs an image obtained by capturing an image of the subject acquired by the image acquiring unit 101 from the side surface, position information about the neck in the image, and position information about the hip joint in the image. Image capturing from the side means image capturing in the sagittal plane. Then, based on the position information about the neck and the hip joint in the image, the candidate point cloud extracting unit 114 designates a rectangular region including at least the back of the subject shown in the image like a bounding box BB exemplified in FIG. 7. The shape of the designated region is not limited to a rectangle. Next, the candidate point cloud extracting unit 114 performs edge extraction processing on the data of the image acquired by the image acquiring unit 101, and extracts a point cloud to be a candidate of the edge point cloud.

More specifically, the position and size of the bounding box BB are determined based on the position of the neck indicated by the key point P8, the position of the hip joint indicated by the key point P9, and the distance l therebetween in the image. For example, for the side in the direction of a distance l of the bounding box BB, the length l_{m,0} of the bounding box BB from the neck toward the side opposite to the hip joint and the length l_{m,1} of the bounding box BB from the hip joint toward the side opposite to the neck can be calculated by the following two expressions. That is, for the side in a distance l direction, each length can be calculated with l_{m,0}=l×p₀ and l_{m,1}=l×p₁, and the length of the side can be calculated with l_{m,0}+l+l_{m,1}. Furthermore, for the side perpendicular to the direction of the distance l of the bounding box BB, a length including an edge point cloud LMa that is the shape of the back or a length obtained by adding a predetermined distance to the length can be obtained by calculation in a line parallel to the line connecting the neck and the hip joint. p₀,p₁ can be parameters determined by a person.

Furthermore, the edge extraction processing may be applied only to the inside of the bounding box BB, or may be applied to the entire image to use only the edge in the bounding box BB. The edge extraction may use an edge of the person region acquired using a learned machine learning model for the person region segmentation obtained in advance by machine learning.

In addition, the joint position of the knee can also be used as the joint position information. In this case, the size may be determined by defining a bounding box including the joint position of the knee instead of 1 as the distance between the hip joint and the knee. Compared with the distance l between the neck and the hip joint, there is an advantage that the distance between the hip joint and the knee changes less depending on the posture.

As described above, the candidate point cloud extracting unit 114 may input the first image obtained by capturing an image of the sagittal plane of the subject in the upright posture or the forward bending posture, and determine the rectangular region including the back of the subject based on the points indicating the positions of the neck and the hip joint of the subject designated in the first image. The designation in the first image may be automatically executed, or may be designation by the user from the input unit 104. Then, in this case, the candidate point cloud extracting unit 114 extracts the edge candidate point cloud by extracting a point cloud indicating the position of another part in the rectangular region.

As another processing example of the edge extraction, an example that is effective particularly in the case of the upright position or the backward bending posture will be described with reference to FIG. 8. In the case of the upright position or the backward bending posture, the shape of the actual back is often greatly different from the edge of the back surface of the body acquired from the image. Therefore, in addition to the back surface, the edge of the front surface of the body or the center line of the front surface and the back surface is translated to the position of the back surface of the body.

First, also in this example, the candidate point cloud extracting unit 114 inputs an image obtained by capturing an image of the subject acquired by the image acquiring unit 101 from the side surface, position information about the neck in the image, and position information about the hip joint in the image. Then, the candidate point cloud extracting unit 114 sets a rectangular region such as the bounding box BB illustrated in FIG. 7 as a region of interest based on the position information about the neck and the hip joint in the image, as in the case of the forward bending posture. Although not illustrated in FIG. 8, since the rectangular region in this example is in the backward bending posture, the shape is greatly different from the bounding box BB for the forward bending state illustrated in FIG. 7.

Furthermore, the candidate point cloud extracting unit 114 performs edge extraction processing on the data of the image acquired by the image acquiring unit 101, and extracts an edge Fa of the body front surface and an edge Fb of the body back surface as illustrated in FIG. 8. Next, the candidate point cloud extracting unit 114 translates in such a way as to align an edge Fa of the body front surface with a predetermined position of an edge Fb of the body back surface. Alternatively, the candidate point cloud extracting unit 114 may perform edge extraction processing on the data of the image acquired by the image acquiring unit 101 to obtain the center line of the edge as follows. That is, the candidate point cloud extracting unit 114 may calculate the average of the x coordinates for each point in the edge point cloud having the same y coordinate in the edge Fa of the body front surface and the edge Ba of the body back surface illustrated in FIG. 8 to obtain the midpoint, and obtain the point cloud as the center line Ca of the edges of the front surface and the back surface. The center line Ca may be a center line approximated to be a smooth curve with respect to the center line from which the midpoint is obtained in this manner. Next, the candidate point cloud extracting unit 114 translates in such a way as to align a center line Ca with a predetermined position of the edge Fb of the body back surface. In FIG. 8, an example of this translation is illustrated by a thick arrow. In any method, the candidate point cloud extracting unit 114 can extract the edge candidate point cloud using the translation of the extractable edge.

As described above, the candidate point cloud extracting unit 114 may input the first image obtained by capturing an image of the sagittal plane of the subject in the upright posture or the backward bending posture, and detect the back surface edge line indicating the edge of the back surface of the subject and the front surface edge line indicating the edge of the front surface in the first image. Then, the candidate point cloud extracting unit 114 may translate the center line between the back surface edge line and the front surface edge line or the front surface edge line toward the back surface edge line in such a way as to match a part of the back surface edge line. The candidate point cloud extracting unit 114 can extract the edge candidate point cloud by such translation.

### <Variation 2>

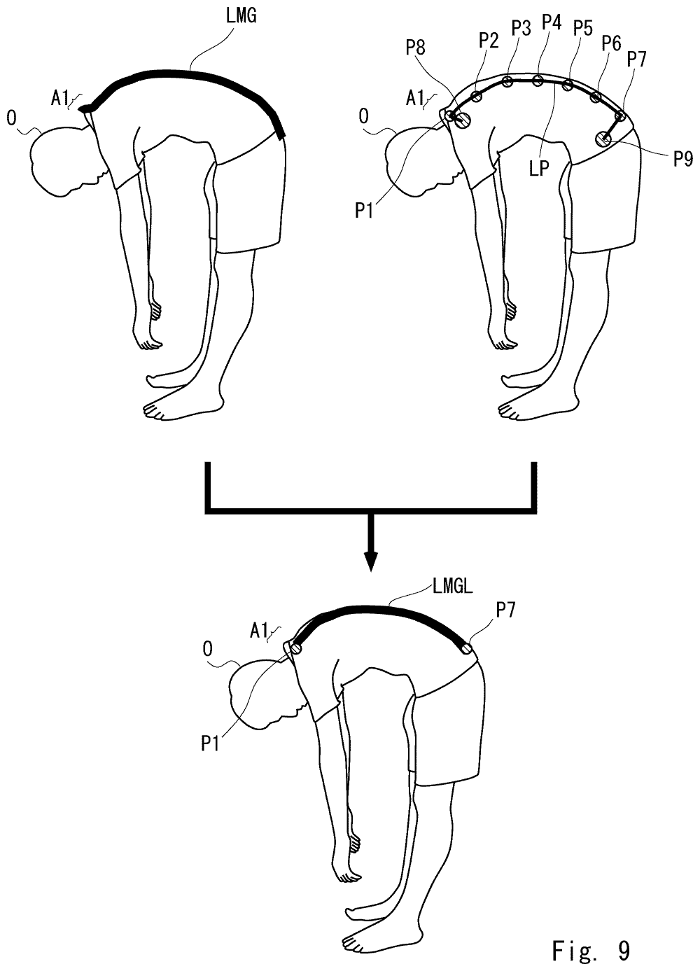

A configuration example in which the back shape estimating unit 105 includes the end point determining unit 115 will be described as variation 2 with reference to FIG. 9. FIG. 9 is a schematic diagram illustrating an example of end point determination processing in the posture measurement apparatus according to the present disclosure.

In variation 2, the back shape estimating unit 105 inputs the edge candidate point cloud and the joint position information, and outputs the optimized back shape model in which the end point is determined. Specifically, first, the end point determining unit 115 uses the parameters of the back shape model optimized by the parameter optimizing unit 113 and the edge candidate point cloud as inputs. For example, the end point determining unit 115 uses an edge candidate point cloud LMG illustrated in FIG. 9, the key points P1 to P9 of each part determined by the optimized back shape model, and the edge candidate point cloud LMG as inputs. The end point determining unit 115 regards the input edge candidate point cloud LMG as a curve, and determines an end point based on the position of each part determined using the back shape model in such a way as to extract only a designated range of the edge candidate point cloud LMG such as the key points P1 to P7 as an edge. In this example, among the points included in the edge candidate point cloud LMG, the point closest to each of the key points P1 and P7 is determined as the end point. If a point having the same coordinates as the key points P1 and P7 is included in the edge candidate point cloud LMG, the end points match the key points P1 and P7. Then, as exemplified by the edge LMGL in FIG. 9, the end point determining unit 115 extracts the edge by cutting out, that is, trimming the edge point cloud between the two end points from the optimized back shape model.

The edge extraction processing only needs to be able to extract an edge between the key points P1 and P7. Therefore, the edge between them may be an edge that completely matches the optimized back shape model, or may be an edge extracted based on the optimized back shape model and the edge candidate point cloud LMG.

Of course, the specified range is not limited thereto. For example, in a case where a region between the thoracic vertebrae 1 and the lumbar vertebra 5 is set as an edge, the end point determining unit 115 determines the position of each part of the spinal columns using the optimized back shape model, and then acquires the positions associated to the thoracic vertebrae 1 and the lumbar vertebra 5. Then, the end point determining unit 115 can acquire points closest to the acquired positions from the edge candidate point cloud, determine the points as end points, and extract designated sections having both end points as the start and end of the section. In addition, intermediate positions of adjacent vertebrae, for example, between the lumbar vertebra 7 and the thoracic vertebra 1 and between the lumbar vertebra 5 and the sacrum can be designated as the positions of the end points. In addition, only a partial section of the spinal column can be extracted as an edge by specifying a part of the spinal column such as between the thoracic vertebrae 1 and the thoracic vertebrae 12.

In this manner, the end point determining unit 115 can input the plurality of optimized parameters and the edge candidate point cloud. Then, the end point determining unit 115 may determine both end points of the curve as both end points of the edge candidate point cloud based on the position of each part configuring the corrected back shape model in such a way as to extract only a designated range of the curve connecting each point of the edge candidate point cloud.

### <Variation 3>

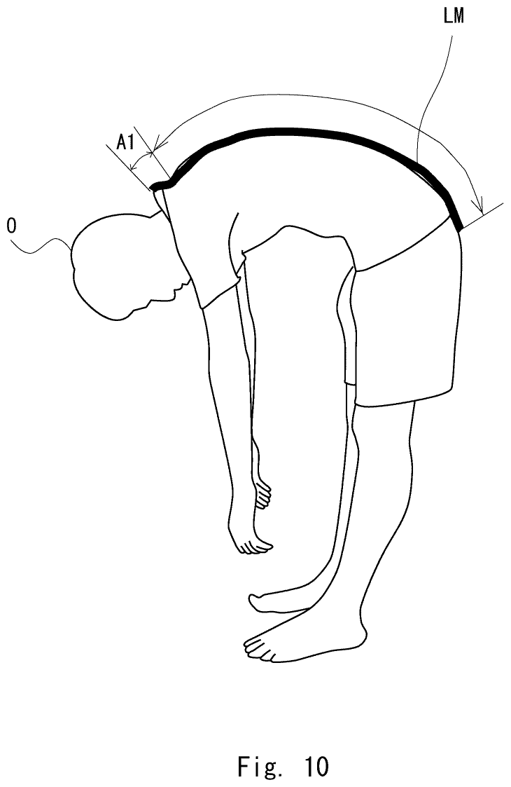

A configuration example in which the back shape estimating unit 105 includes the edge adding unit 116 will be described as variation 3 with reference to FIG. 10. The edge adding unit 116 can also be simply referred to as an adding unit. FIG. 10 is a schematic diagram illustrating an example of edge addition processing in the posture measurement apparatus according to the present disclosure.

In Variation 3, the back shape estimating unit 105 uses the edge candidate point cloud and the joint position information as inputs, and outputs an optimized and weighted average back shape model. Specifically, first, the edge adding unit 116 inputs the parameters of the optimized back shape model, the edge candidate point cloud or the edge point cloud trimmed by the end point determining unit 115, and the weighting factor associated thereto. Then, the edge adding unit 116 gives a weight to each of the edge point cloud calculated from the back shape model and the edge point cloud input or acquired from the image for each predetermined section, takes a weighted average for each point, and outputs an averaged edge point cloud.

For example, in the input edge candidate point cloud LM illustrated in FIG. 10, the edge cannot be accurately acquired in the portion of a region A1 on the head side with respect to the position on the left side in the range indicated by the double arrow, but the edge can be relatively accurately acquired in the section of the range indicated by the double arrow. In such a case, the influence of the estimation error of the parameter potentially present in the back shape model can be reduced by taking the weighted average of the edge and the candidate point cloud calculated using the back shape model. That is, if the accuracy of the estimation by the back shape model is lower than the accuracy of the edge candidate point cloud, the edge point cloud may be determined with emphasis on the weight of the edge candidate point cloud. For reference, the region A1 is also illustrated in FIGS. 3 to 5 and 9.

As described above, the edge adding unit 116 inputs the plurality of parameters corrected by the parameter optimizing unit 113 and the edge candidate point cloud. Then, the edge adding unit 116 adds a predetermined weight to each point of the point cloud indicating the position of each part configuring the back shape model indicated by the plurality of corrected parameters and the candidate point of the edge candidate point cloud associated to each point to obtain a weighted average. In this manner, the edge adding unit 116 obtains a point cloud representing the shape of the back of the subject.

### <Variation 4>

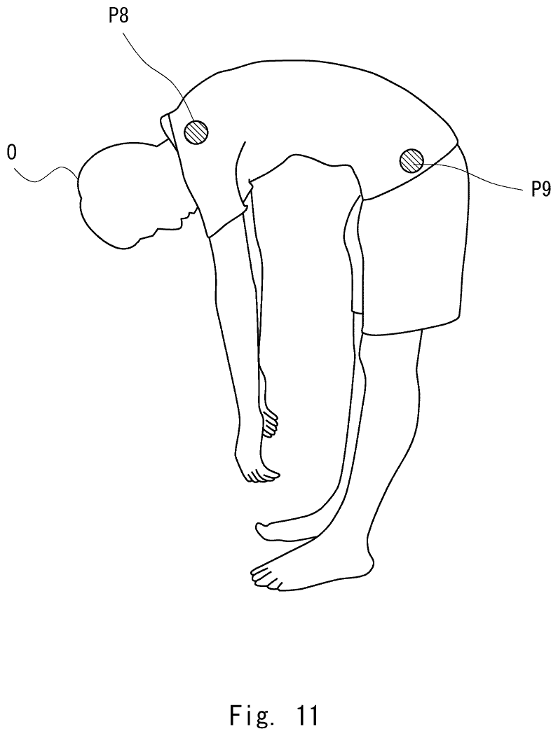

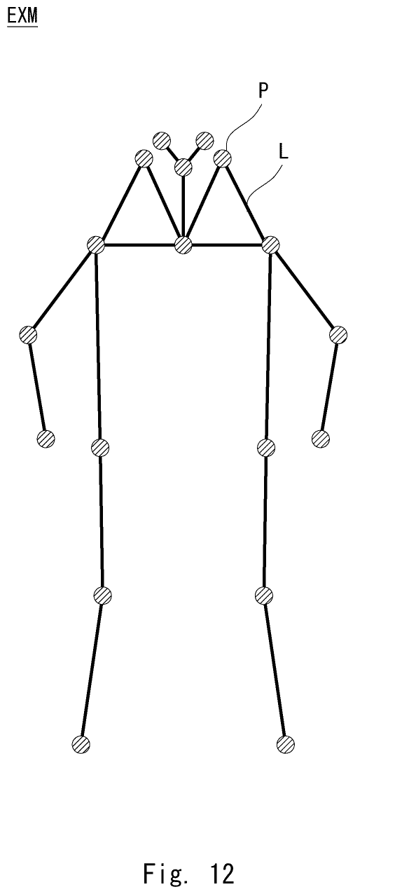

A configuration example in which the posture measurement apparatus 100 includes the body keypoint extracting unit 103 will be described as variation 4 with reference to FIGS. 11 and 12. FIG. 11 is a schematic diagram illustrating an example of body keypoint extraction processing in the posture measurement apparatus according to the present disclosure. FIG. 12 is a schematic diagram illustrating an example of body keypoints extracted in the body keypoint extraction processing in the posture measurement apparatus according to the present disclosure.

In variation 4, the back shape estimating unit 105 separately inputs the edge candidate point cloud input by the user from the input unit 104 and the key point position information indicating the position of the body keypoint extracted by the body keypoint extracting unit 103, and outputs the optimized back shape model. Therefore, first, the body keypoint extracting unit 103 extracts the key point position information from the image acquired by the image acquiring unit 101.

For example, the body keypoint extracting unit 103 extracts key point information indicating at least the positions of the neck and the hip joint, such as the key points P8 and P9 in FIG. 11, by estimating the key point information from the image. For example, the body keypoint extracting unit 103 extracts the key point information from the image using a learned body keypoint extraction model machine-learned in such a way as to input an image obtained by capturing an image of the subject from the side surface from the image acquiring unit 101 and to output key point information indicating a predetermined body keypoint cloud. Again, the skeletal key points indicating the position of the neck can be extracted as skeletal key points indicating the position of the predetermined vertebrae. For example, the position of the neck can be estimated as the position of the ridge, which is the cervical vertebra 7, among the seven vertebrae. Alternatively, the position near the middle between the cervical vertebra 7 and the sternum stalk, which is estimated as the key point of the “neck” in the general body keypoint extraction model, can be estimated as the position of the neck.

The key point information in the body keypoint extracting unit 103 can be extracted as information indicating the body keypoint from the image using the existing body keypoint extraction model expressed by the plurality of key points P and the connection portion L between the adjacent key points P as illustrated in FIG. 12. However, the method of extracting the key point information in the body keypoint extracting unit 103 is not limited to this, for example, the method disclosed in Patent Literature 1 is adopted.

In this manner, the body keypoint extracting unit 103 may extract the key point position information from the second image by using the learned model machine-learned in such a way as to input the second image obtained by capturing an image of the subject from the side surface and output the key point position information indicating the position of the body keypoint. Then, the back shape estimating unit 105 may determine a plurality of parameters of the back shape model based on the extracted key point position information, and the back shape model may be updated according to this determination. Thereafter, this back shape model is optimized.

### <Variation 5>

In variation 5, the posture measurement apparatus 100 includes a feature calculating unit 106, and extracts a feature related to a state of each part of the subject from the shape of the back estimated by the back shape estimating unit 105. The feature calculating unit 106 can be referred to as a feature extracting unit in order to extract a feature. In variation 5, the configuration of the back shape estimating unit 105 is not limited, and any variation of the configuration may be adopted. It can be said that the posture measurement apparatus 100 functions as a posture evaluation apparatus since the feature indicating the posture can be output by including the feature calculating unit 106.

The feature calculating unit 106 uses an edge point cloud or an edge point cloud and joint position information as inputs. Here, the input joint position information can include position information indicating positions of key points other than the neck and the hip joint, for example, key points of the shoulder, the knee, and the ankle joint. Then, the feature calculating unit 106 extracts a feature representing the state of each body part with a numerical value based on the input information, and outputs the extracted feature. The output destination may be the display unit 108 or the state estimating unit 107.

The extracted feature may be, for example, an amount representing the curvature of the edge point cloud, that is, the shape of the back that appears if the same posture is taken in a state where no clothing is worn. Specifically, the curvature is calculated for all the points included in the edge point cloud by fitting an nth-order function or a spline function to the edge point cloud. Since the position of each part of the spinal column can be specified by the back shape model, the feature calculating unit 106 can identify the part, for example, the average of the curvatures between the thoracic vertebrae 1 to 6, and calculate the representative value of the curvature in units of sections. In addition, in the detailed model exemplified in FIG. 4, all the vertebrae from the ridge to the sacrum can be included, and for example, a representative value of the curvature can be calculated between the vertebrae, such as an average value or a median value of the curvatures of the cervical vertebrae 7 and the thoracic vertebrae 1.

Furthermore, as the extracted feature, for example, the angle of each part or the relative angle between the parts, which is one of the parameters of the back shape model, can be used as it is. As another method of extracting the feature, various methods such as the method disclosed in Patent Literature 1 can be used.

In this manner, the feature calculating unit 106 may extract the feature regarding the state of each part of the subject indicated by the shape of the back of the subject based on the point cloud indicating the position of each part configuring the back shape model indicated by the plurality of parameters corrected by the parameter optimizing unit 113.

### <Variation 6>

In Variation 6, the posture measurement apparatus 100 includes a feature calculating unit 106 and a state estimating unit 107, extracts a feature related to the state of each part of the subject from the shape of the back estimated by the back shape estimating unit 105, and estimates the state based on the extracted feature. In variation 6, the configuration of the back shape estimating unit 105 is not limited, and any variation of the configuration may be adopted. The posture measurement apparatus 100 functions as a posture evaluation apparatus by including the feature calculating unit 106 and the state estimating unit 107.

The state estimating unit 107 estimates the state of each part of the subject based on the feature extracted by the feature calculating unit 106, and outputs the estimated state. The output destination may be the display unit 108. For example, the state estimating unit 107 inputs the feature extracted by the feature calculating unit 106 as described in variation 5, and evaluates the state of each part using a list of reference values for the feature and a learned machine learning model. This list can be stored in a storage unit (not illustrated). In addition, a machine learning model can also be stored in a storage unit (not illustrated), and the same applies to the machine learning models described in other variations.

For example, in evaluating the state of the upper thoracic vertebrae, the state estimating unit 107 compares the average of the curvatures between the thoracic vertebrae 1 to 6 with a reference value for the average in the list. A list of the reference values and a machine learning model may be prepared for each posture such as a forward bending posture, a backward bending posture, and an upright posture. In this case, the determination of the posture may be performed using another machine learning model in which the user inputs the posture or determines the posture from the feature. In addition, various methods such as the method disclosed in Patent Literature 1 can be used for state estimation in the state estimating unit 107.

As a result, the state estimating unit 107 can output any one of a state of insufficient bending, a state of moderate bending, and a state of excessive bending for each part in the forward bending posture. In addition, the state estimating unit 107 can output any one of a state of insufficient extension, a state of moderate extension, and a state of excessive extension for each part in the case of the backward bending posture. In addition, in the upright position, the state estimating unit 107 can output any state of insufficient lordosis, moderate lordosis, and excessive lordosis, or any state of insufficient lordosis, moderate lordosis, and excessive lordosis for each part. In addition, in the case of the upright position, the thoracic vertebrae are kyphosis and the lumbar vertebrae are lordosis. Therefore, the state estimating unit 107 can output a value or the like obtained by evaluating whether each degree is appropriate. The expression of the state is an example, and the three-stage output or the different expression may be used, or any two-stage evaluation of appropriate and inappropriate or a value obtained by scoring a state from insufficiency to excess can be output.

### <Variation 7>

In Variation 7, the posture measurement apparatus 100 includes an input unit 104, and outputs information input by the input unit 104 to the back shape estimating unit 105.

The input unit 104 can receive the position information about the body keypoint as an input from the user. In particular, in a configuration in which the posture measurement apparatus 100 does not include the body keypoint extracting unit 103, it is possible to receive position information about body keypoints as an input from the user and pass the position information to the back shape estimating unit 105. In this example, the configuration of the back shape estimating unit 105 is not limited, and any variation of the configuration may be adopted.

The position information about the body keypoint can be received, for example, as coordinates in a two-dimensional coordinate space in which one point in the sagittal plane of the subject is set as an origin and each of the vertical direction (the downward direction is positive) and the horizontal direction (the direction from the front surface toward the back surface of the subject is positive) is set as one axis. Since the joint is in a three-dimensional space, the input unit 104 may be configured to input three-dimensional coordinates. In this case, the coordinates orthogonally projected on the sagittal plane may be used for the subsequent processing.

Furthermore, in a case where the input in the posture measurement apparatus 100 includes the image acquired by the image acquiring unit 101, the input unit 104 may receive the position information as follows. That is, the input unit 104 may receive, as the position information about the body keypoint, the coordinate information on the two-dimensional plane stretched by two axes in which the downward direction of the image is positive and the rightward direction of the image is positive with the upper left of the input image as the origin. Of course, the input unit 104 may receive only the numerical information about the coordinates, or the input unit 104 and the display unit 108 may be integrated to provide the position information about the body keypoint on the image displayed on the display unit 108 using a mouse cursor, a touch panel, or the like.

Furthermore, in the posture measurement apparatus 100, by including or connecting a measurement system capable of acquiring coordinates in a three-dimensional space such as a motion capture system, position information about a body keypoint can also be input as three-dimensional coordinates. Furthermore, the posture measurement apparatus 100 can include a system in which a head mounted display with a camera (HMD) capable of self-position estimation and hand tracking are combined, or can be connected to the system. As a result, the input unit 104 can also input the position information about the body keypoint while the evaluator such as a therapist palpates the body of the subject while wearing the HMD. In this case, the HMD can also serve as the display unit 108. Furthermore, in a case where three-dimensional coordinates can be acquired, the image acquired by the image acquiring unit 101 is not limited to a two-dimensional image, and may also have three-dimensional point cloud information representing the body surface of the subject. Furthermore, the position information about the body keypoint may be a three-dimensional polygon model representing the body surface or relative position information with respect to the surface model, which is converted from the point cloud information by a conversion unit (not illustrated).

As exemplified by the input unit 104, the posture measurement apparatus 100 can include a key point input unit that inputs key point position information indicating the position of the body keypoint by a user operation. In this case, the back shape model holding unit 112 may determine a plurality of parameters to be held based on the input key point position information.

The input unit 104 can also receive an edge candidate point cloud as an input from the user. In particular, in a configuration in which the back shape estimating unit 105 does not include the candidate point cloud extracting unit 114, it is also possible to receive an edge candidate point cloud as an input from the user and pass the edge candidate point cloud to the back shape estimating unit 105. The input for each point included in the edge candidate point cloud may be received by the same method as the position information about the body keypoint. In this example, as long as the configuration of the back shape estimating unit 105 does not include the candidate point cloud extracting unit 114, any variation of the configuration may be adopted. However, even in the configuration including the candidate point cloud extracting unit 114, the input unit 104 may be used to correct the edge candidate point cloud extracted by the candidate point cloud extracting unit 114.

As exemplified by the input unit 104, the posture measurement apparatus 100 can include a candidate point cloud input unit that inputs an edge candidate point cloud by a user operation.

### <Variation 8>

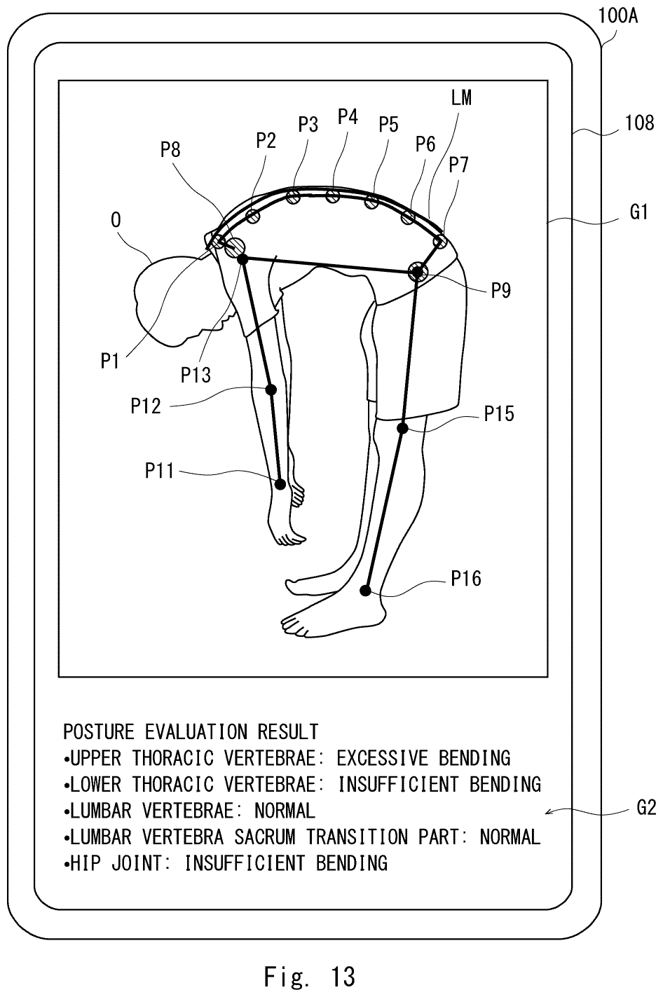

With reference to FIG. 13, a configuration example in which posture measurement apparatus 100 includes display unit 108 will be described as variation 8. FIG. 13 is a diagram illustrating an example of an image displayed on the display unit in the posture measurement apparatus according to the present disclosure. A posture measurement apparatus 100A illustrated in FIG. 13 is an example in which the posture measurement apparatus 100 illustrated in FIG. 2 includes a tablet terminal.

In Variation 8, the posture measurement apparatus 100 such as the posture measurement apparatus 100A includes the display unit 108, generates images indicating various output results and progress, and displays the generated images on the display unit 108. Therefore, the posture measurement apparatus 100 may include an image generation unit (not illustrated). In variation 8, a configuration of any variation may be adopted regardless of other configurations of the posture measurement apparatus 100.

The display unit 108 can display, for example, an image obtained by superimposing a measurement result or an evaluation result on an acquired image such as the image G1 illustrated in FIG. 13, or an image including information indicating a posture evaluation result such as the image G2. An example in which the joint position information indicated by the key points P1 to P9 and P11 to P16 and the back shape model including the edge point cloud LM are displayed in a superimposed manner is illustrated in the image G1. In this manner, the display unit 108 can display the point cloud indicating the position of each part configuring the back shape model indicated by the plurality of parameters corrected by the parameter optimizing unit 113. In the image G2, an example is described in which information indicating the state of each part is displayed as the posture evaluation result. However, examples of the information displayed on the display unit 108 are not limited thereto, and various information such as a value of the feature and a temporal change of the feature can be displayed.

The temporal change of the feature refers to a change of the feature calculated for each of the images acquired by the image acquiring unit 101 every lapse of time. The image acquired by the image acquiring unit 101 is assumed to be a still image, but it is also possible to process each frame using a moving image or an image sequence cut out from the moving image as an input. In this case, since the feature is calculated by the number of frames, the temporal change of the feature can be displayed as a graph in which the horizontal axis is time, or the angle of the trunk with respect to the thigh, and the vertical axis is the value of the feature. For example, at the time where the image acquiring unit 101 acquires a moving image, by displaying a change in curvature, which is one of the features, for each part, the start and end timings of the movement can be known as a time point at which the increase or decrease in curvature starts. Based on this, it is possible to evaluate that a part that starts moving early has high flexibility and a part that stops moving earlier has low flexibility.

### <Variation 9>

In Variation 9, the posture measurement apparatus 100 includes the image acquiring unit 101, and outputs the image acquired by the image acquiring unit 101 to the body keypoint extracting unit 103 or the candidate point cloud extracting unit 114. As described in variation 8, the posture measurement apparatus 100 can also output necessary information to the display unit 108 by superimposing the necessary information on the image acquired by the image acquiring unit 101. In addition, the posture measurement apparatus 100 can function as a device that automatically measures the posture from the image capturing by the configuration including the body keypoint extracting unit 103 or the candidate point cloud extracting unit 114 and the image acquiring unit 101.

### <Variation 10>

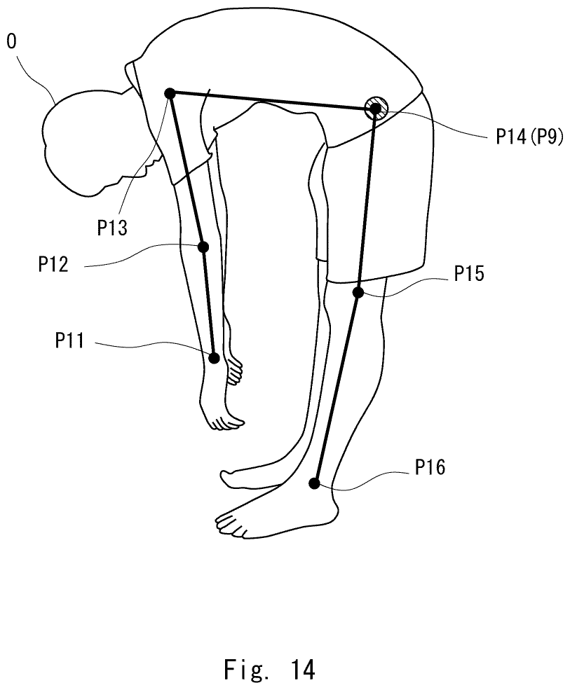

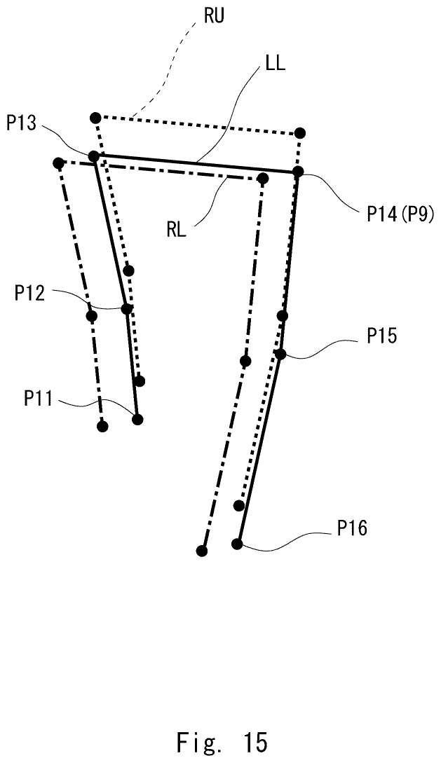

A configuration example in which the posture measurement apparatus 100 includes the image acquiring unit 101 and the imaging angle determining unit 102 will be described as a variation 10 with reference to FIGS. 14 and 15. FIGS. 14 and 15 are schematic diagrams illustrating an example of imaging angle determination processing in the posture measurement apparatus according to the present disclosure.

The imaging angle determining unit 102 uses the image acquired by the image acquiring unit 101 or the position information extracted by the body keypoint extracting unit 103 for the image as an input, and determines the angle at the time of image capturing with respect to the input image or position information. The angle at the time of image capturing can be an angle of an optical axis of an image capturing apparatus such as a camera. The determination is performed by estimating the angle of the optical axis of the image capturing apparatus with respect to the sagittal plane using the learned machine learning model. The angle output as the determination result is the angle of the image capturing apparatus with respect to the sagittal plane, but for example, in a case where it indicates that the angle is greatly away from the vertical direction, a warning can be returned.

The imaging angle determining unit 102 may output a line or the like indicating the angle at the time of capturing an image to the display unit 108 in such a way as to be superimposed on the image acquired by the image acquiring unit 101 and display the line or the like. As a result, as illustrated in FIG. 13, in a case where an image is captured from a direction perpendicular to the sagittal plane, body keypoints existing on the left and right sides appear to overlap each other. Here, only body keypoints P11 to P16 are illustrated.

On the other hand, if an image is captured looking down from above with respect to the sagittal plane, a key point cloud RU including the right body keypoints appears as compared with a key point cloud LL including the left body keypoints P11 to P16 in FIG. 14. In a case where an image is captured in a direction from slightly left to diagonally right with respect to the sagittal plane, a key point cloud RL including right body keypoints appears to be indicated with respect to a key point cloud LL on the left side in FIG. 14. As described above, in a case where the image is captured from a direction deviated from the direction perpendicular to the sagittal plane, the key points existing on the left and right appear to be deviated in a certain direction. In the machine learning model used for the determination, the machine learning may be performed such that the angle of the optical axis, which is the capturing angle, is estimated using the deviation of the position information about the body keypoints such as the shoulders and the hip joints existing on both the right and left sides as an input.

In this manner, the imaging angle determining unit 102 may input the second image obtained by capturing the sagittal plane of the subject or the key point position information indicating the position of the body keypoint extracted from the second image, and may estimate the angle of the optical axis from the input using the machine learning model. This machine learning model is a learned model subjected to machine learning in such a way as to input the second image or the key point information and output the angle of the optical axis with respect to the sagittal plane at the time that the second image is captured.

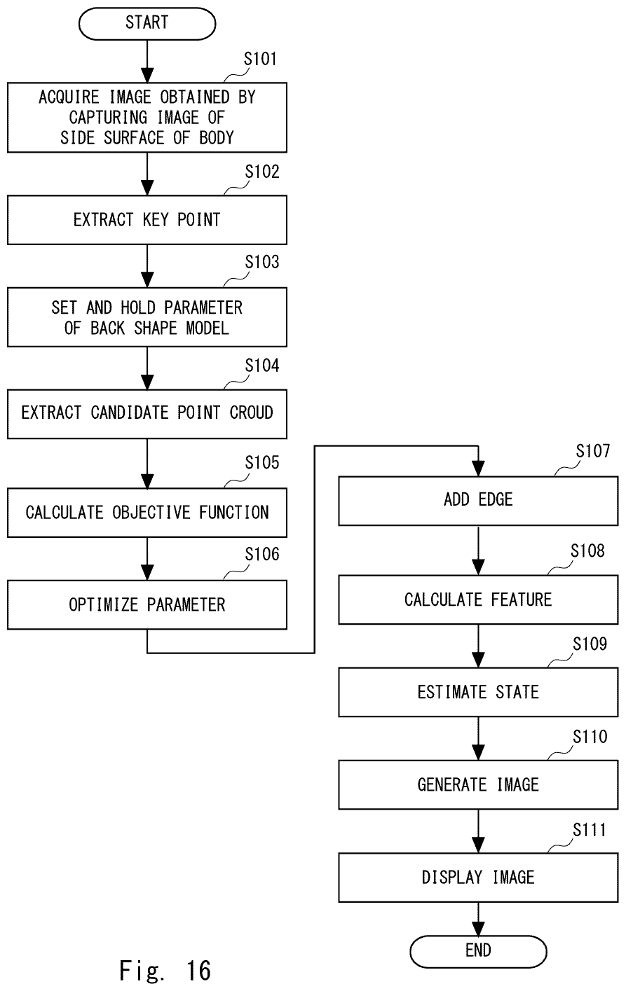

Next, a posture evaluation method executed by the posture measurement apparatus 100 will be described with reference to FIG. 16. FIG. 16 is a flowchart illustrating a posture evaluation method according to the present disclosure. Although only a simple flow will be described here, various examples described above can be applied. Therefore, the following posture evaluation method can also be an example of a posture measurement method that does not perform posture evaluation by applying some examples described above.

First, the image acquiring unit 101 acquires an image obtained by imaging the side surface of the body (step S101), and inputs the obtained image to the body keypoint extracting unit 103. Next, the body keypoint extracting unit 103 extracts key point position information indicating the positions of the body keypoints from the image acquired in step S101 (step S102).

Next, the back shape model holding unit 112 sets or updates parameters configuring each part based on the key point information extracted by the body keypoint extracting unit 103, and holds the set or updated back shape model (step S103).

Next, the candidate point cloud extracting unit 114 extracts a point cloud that becomes a candidate of the edge point cloud, that is, the edge candidate point cloud based on the image acquired by the image acquiring unit 101 and the position information indicating the positions of the neck and the hip joint in the image (Step S104). The order of steps S102 to S103 and step S104 is not limited.

Next, the objective function calculating unit 111 inputs the extracted edge candidate point cloud and the held back shape model, calculates a value of a predetermined objective function, and outputs the value to the parameter optimizing unit 113 (step S105). The parameter optimizing unit 113 optimizes the parameters by correcting a plurality of parameters in the back shape model in such a way as to minimize a value of a predetermined objective function using a predetermined constraint condition and the held back shape model as inputs (step S106).

Next, the edge adding unit 116 inputs the parameters of the optimized back shape model, the edge candidate point cloud or the edge point cloud after trimming determined in advance by the end point determining unit 115, and the weighting factor associated thereto, and adds the edges (step S107). In step S107, the edge adding unit 116 gives a weight to each of the edge point cloud calculated from the back shape model and the edge point cloud input or acquired from the image for each predetermined section, and takes a weighted average for each point. The edge adding unit 116 outputs the edge point cloud thus averaged to the feature calculating unit 106.

Next, the feature calculating unit 106 extracts a feature based on the input edge point cloud and outputs the feature to the state estimating unit 107 (step S108). The state estimating unit 107 estimates a state of a posture of, for example, the upper thoracic vertebrae, the lower thoracic vertebrae, the lumbar vertebrae, or the like based on the feature input from the feature calculating unit 106 (step S109). Then, the state estimating unit 107 or an image generation unit (not illustrated) generates an image indicating the estimation result (step S110) and passes the image to the display unit 108. The display unit 108 displays an image indicating the estimation result (step S111), and ends the processing.

The present example embodiment has been described above. Also in the present example embodiment, similarly to the first example embodiment, even in a scene where the shape of the back of the subject cannot be accurately grasped due to the presence of the clothes of the subject, the shape of the back of the subject can be simply and accurately measured.

For example, according to the present example embodiment, by using the back shape model, it is possible to prevent estimation that cannot occur in terms of the structure of the body, and thus, it is possible to improve the estimation accuracy of the edge. According to the present example embodiment, even if the shape of the body is hidden by the clothing, the shape of the back can be accurately estimated, and for example, the accuracy of the evaluation for each part can be improved even in the forward bending posture of a person wearing a loose T-shirt and bending forward.

Furthermore, according to the present example embodiment, for example, even in a case where only a two-dimensional image obtained by a camera such as a smartphone is used, the shape of the back can be accurately estimated, and the posture can be accurately evaluated based on the estimation. In fact, with the spread of online training and self-training, there is an increasing need for ordinary people to evaluate their own posture and alignment, but the present example embodiment can also respond to such needs. That is, even a person who is not an expert such as a trainer or a therapist can estimate the shape of the back and evaluate the state relatively easily and accurately using a two-dimensional image obtained by a camera such as a smartphone in a scene of online training, self-training, or the like. In addition, the present example embodiment can also be used for simple screening before treatment of a subject by a trainer, a therapist, or the like.

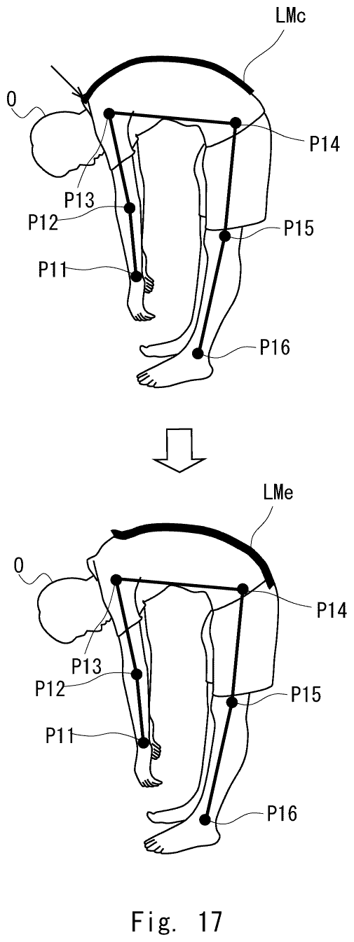

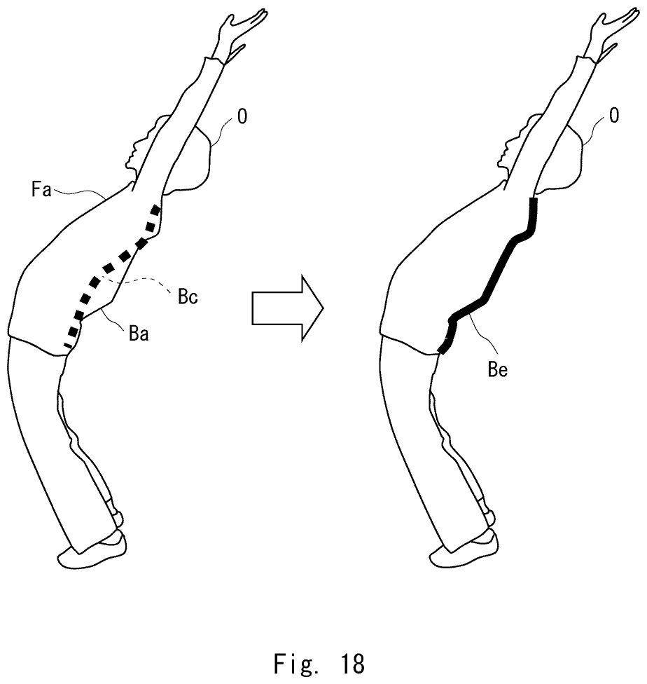

In order to supplement the effects of the present example embodiment, comparative examples illustrated in FIGS. 17 and 18 will be described. FIG. 17 is a diagram illustrating an estimation result of the shape of the back in the forward bending posture according to the comparative example. FIG. 18 is a diagram illustrating a result of estimation of the shape of the back in the backward bending posture according to the comparative example.

In the comparative example illustrated in FIG. 17, an example in which the shape of the back cannot be well estimated because the end point cannot be accurately estimated will be described. As indicated by an edge point cloud LMc in the diagram illustrated on the upper side of FIG. 17, there is a case where an edge point cloud indicating the shape of the back should be estimated correctly, but cannot be estimated well as indicated by an edge point cloud LMe in the diagram illustrated on the lower side of FIG. 17. It can be seen that the edge point cloud LMe does not continue to the portion indicated by the arrow in the vicinity of the left end of the edge point cloud LMc. This occurs because the subject wears thick clothes such as a parker, and a slack portion such as a hood of the parker hides the shape of the body, in such a way that it cannot be accurately estimated. On the other hand, in the present example embodiment, since the end points can be appropriately determined, it is possible to estimate an edge point cloud indicating the shape of the back correctly like the edge point cloud LMc as the shape of the back that appears if the same posture is taken in a state where the clothing is not worn.

In the comparative example illustrated in FIG. 18, an example in which the shape of the back cannot be well estimated in the backward bending posture due to the sagging and wrinkles of the clothing is exemplified. As indicated by the edge point cloud Bc indicating the shape of the back in the diagram illustrated on the left side of FIG. 18, there is a case where the edge point cloud indicating the shape of the back should be estimated correctly even in the backward bending posture but cannot be estimated well as indicated by the edge point cloud Be in the diagram illustrated on the right side of FIG. 18. The edge point cloud Be indicates an edge point cloud according to a comparative example in which an edge extraction method for separating a person and a background is adopted, and in such an edge extraction method, an edge of clothes is extracted instead of an edge of the back. On the other hand, in the present example embodiment, since the edge point cloud is estimated by complementarily using the information about the body front surface as described above, it is possible to estimate a relatively accurate edge point cloud as indicated by the edge point cloud Bc as the shape of the back that appears if the same posture is taken in a state where the clothing is not worn.

In addition, in the comparative example illustrated in either of FIGS. 17 and 18, the shape of the edge is used for the calculation in the extraction of the feature, which is the processing in the subsequent stage. Therefore, as in the edge point cloud LMe in the comparative example of FIG. 17, the feature cannot be correctly extracted because the edge point is shifted, that is, the edge point is shifted from the body part to be evaluated. In addition, as in the edge point cloud Be in the comparative example of FIG. 18, since the shape of the back is not accurate, the feature cannot be correctly extracted. On the other hand, in the present example embodiment, the feature can be accurately extracted, and the state in the subsequent stage can be accurately estimated.

### Third Example Embodiment

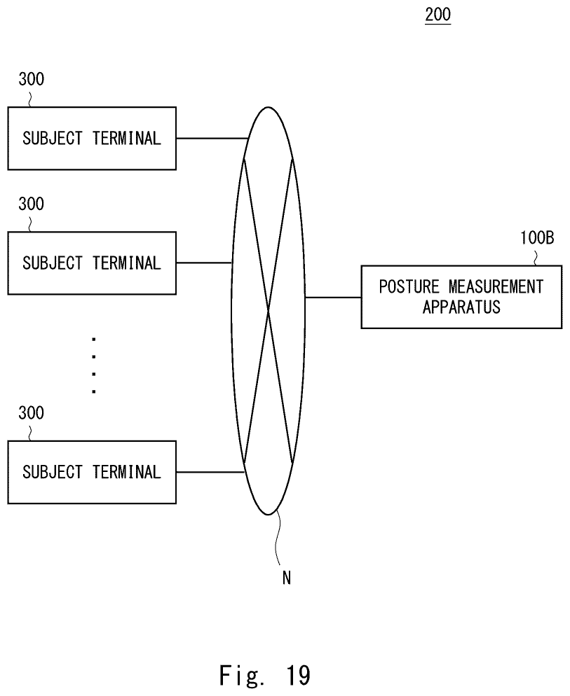

Next, a configuration example of a posture evaluation system 200 will be described with reference to FIG. 19. FIG. 19 is a diagram illustrating a configuration example of a posture evaluation system according to the present disclosure. As illustrated in FIG. 19, the posture evaluation system 200 includes a posture measurement apparatus 100B and a subject terminal 300 communicable with the posture measurement apparatus 100B. The posture measurement apparatus 100B includes the main components described in the posture measurement apparatus 100. The posture measurement apparatus 100B and the subject terminal 300 can communicate with each other via the network N. Furthermore, as illustrated in FIG. 19, one or more subject terminals 300 may be able to communicate with the posture measurement apparatus 100B. The subject terminal 300 is a smartphone, a tablet terminal, a personal computer, or the like possessed by the subject.

The posture measurement apparatus 100B acquires an image obtained by capturing an image of a side surface of a body of a subject whose posture is to be measured or evaluated from the subject terminal 300. Therefore, the posture measurement apparatus 100B is different from the posture measurement apparatus 100 in FIG. 2 in that the image acquiring unit 101 does not need to include an image capturing apparatus. Furthermore, the estimation result display image generated for display on the display unit 108 by the posture measurement apparatus 100B may be transmitted to the subject terminal 300 and displayed on a display unit (not illustrated) of the subject terminal 300.

The subject terminal 300 includes an image capturing apparatus (not illustrated) that images a side surface of the body of the subject whose posture is to be measured or evaluated. The subject terminal 300 transmits the image to the posture measurement apparatus 100B.

### Other Example Embodiments

In each of the above-described example embodiments, the present disclosure has been described as a hardware configuration, but the present disclosure is not limited thereto. The present disclosure can also be achieved by causing a processor such as a central processing unit (CPU) to execute a computer program, for example, the processing procedure described in the flowchart of FIG. 16 and other processing procedures described in the example embodiment.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

While the present disclosure has been particularly shown and described with reference to example embodiments thereof, the present disclosure is not limited to these example embodiments. It will be understood by those of ordinary skill in the art that various changes in form and details may be made therein without departing from the sprit and scope of the present disclosure as defined by the claims. And each embodiment can be appropriately combined with at least one of embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

Some or all of the above-described example embodiments may be described as in the following Supplementary Notes, but are not limited to the following Supplementary Notes.

### (Supplementary Note 1)

A posture measurement apparatus including:
a holding unit that holds a shape model representing a shape of a back of a subject as a function of a plurality of parameters;
a calculating unit that calculates a value of a predetermined objective function using a candidate point cloud as a candidate of a point cloud indicating the shape of the back of the subject and the shape model as inputs; and
an optimizing unit that uses a constraint condition of the parameters and the shape model as inputs and corrects the parameters in such a way as to minimize a value of the predetermined objective function.

### (Supplementary Note 2)

The posture measurement apparatus according to Supplementary Note 1, in which
the shape model is a model representing a shape of the back of the subject in a sagittal plane, and
the parameters includes at least a size of each part configuring the shape model, an angle representing a posture in a sagittal plane, and position information indicating positions of a neck and a hip joint in the sagittal plane.

### (Supplementary Note 3)

The posture measurement apparatus according to Supplementary Note 2, in which the constraint condition of the parameters includes a condition that the position information as a part of the parameters and candidate position information indicating positions of the neck and the hip joint in the sagittal plane as the candidate point cloud match within a predetermined error range.

### (Supplementary Note 4)

The posture measurement apparatus according to any one of Supplementary Notes 1 to 3, in which the calculating unit calculates, as the value of the predetermined objective function, a sum of distances between each part indicated by the shape model and a closest point in the candidate point cloud.

### (Supplementary Note 5)

The posture measurement apparatus according to any one of Supplementary Notes 1 to 3, in which the calculating unit calculates, as the value of the predetermined objective function, a weighted sum of distances between each part indicated by the shape model and a closest point in the candidate point cloud.

### (Supplementary Note 6)

The posture measurement apparatus according to any one of Supplementary Notes 1 to 3, in which the calculating unit calculates, as the value of the predetermined objective function, a sum of the distances between each point of an approximation candidate point cloud obtained by approximating the candidate point cloud based on each part indicated by the shape model and a closest point in the candidate point cloud.

### (Supplementary Note 7)

The posture measurement apparatus according to any one of Supplementary Notes 1 to 3, in which the calculating unit calculates, as the value of the predetermined objective function, a weighted sum of the distances between each point of an approximation candidate point cloud obtained by approximating the candidate point cloud based on each part indicated by the shape model and a closest point in the candidate point cloud.

### (Supplementary Note 8)

The posture measurement apparatus according to any one of Supplementary Notes 1 to 7, further including a candidate point cloud extracting unit that extracts the candidate point cloud by inputting a first image obtained by capturing an image of a side surface of the subject in an upright posture or a forward bending posture, determining a region including a back of the subject based on points indicating positions of a neck and a hip joint of the subject designated in the first image, and extracting a point cloud indicating positions of other parts in the region.

### (Supplementary Note 9)

The posture measurement apparatus according to any one of Supplementary Notes 1 to 7, further including a candidate point cloud extracting unit that extracts the candidate point cloud by inputting a first image obtained by capturing an image of a side surface of the subject in an upright posture or a backward bending posture, detecting a back surface edge line indicating an edge of a back surface of the subject and a front surface edge line indicating an edge of a front surface in the first image, and translating a center line between the back surface edge line and the front surface edge line or the front surface edge line toward the back surface edge line in such a way as to match a part of the back surface edge line.

### (Supplementary Note 10)

The posture measurement apparatus according to any one of Supplementary Notes 1 to 9, further including an end point determining unit that inputs the parameters corrected in the optimizing unit and the candidate point cloud, and determines both end points of a curve connecting the points of the candidate point cloud as both end points of the candidate point cloud based on positions of parts configuring the corrected shape model in such a way as to extract only a designated range of the curve.

### (Supplementary Note 11)

The posture measurement apparatus according to any one of Supplementary Notes 1 to 10, further including an adding unit that obtains a point cloud representing the shape of the back of the subject by inputting the parameters corrected in the optimizing unit and the candidate point cloud, and obtaining a weighted average by adding a predetermined weight to each point of the point cloud indicating the position of each part configuring the shape model indicated by the corrected parameters and the candidate point of the candidate point cloud associated to each point.

### (Supplementary Note 12)

The posture measurement apparatus according to any one of Supplementary Notes 1 to 11, further including a body keypoint extracting unit that extracts key point position information from a second image using a learned model machine-learned in such a way as to input the second image obtained by capturing an image of a side surface of the subject and output the key point position information indicating a position of a body keypoint.

### (Supplementary Note 13)

The posture measurement apparatus according to any one of Supplementary Notes 1 to 12, further including a feature extracting unit that extracts a feature regarding a state of each part of the subject indicated by the shape of the back of the subject based on a point cloud indicating a position of each part configuring the shape model indicated by the parameters corrected by the optimizing unit.

### (Supplementary Note 14)

The posture measurement apparatus according to Supplementary Note 13, further including a state estimating unit that estimates a state of each part of the subject based on the feature extracted by the feature extracting unit.

### (Supplementary Note 15)

The posture measurement apparatus according to any one of Supplementary Notes 1 to 14, further including a key point input unit that inputs key point position information indicating a position of a body keypoint by a user operation,
in which the holding unit determines the parameters to be held based on the input key point position information.

### (Supplementary Note 16)

The posture measurement apparatus according to any one of Supplementary Notes 1 to 15, further including a candidate point cloud input unit that inputs the candidate point cloud by a user operation.

### (Supplementary Note 17)

The posture measurement apparatus according to any one of Supplementary Notes 1 to 16, further including a display unit that displays a point cloud indicating a position of each part configuring the shape model indicated by the parameters corrected by the optimizing unit.

### (Supplementary Note 18)

The posture measurement apparatus according to any one of Supplementary Notes 1 to 17, further including an imaging angle determining unit that estimates an angle of an optical axis from a second image or a key point position information by using a learned model machine-learned in such a way as to input the second image obtained by capturing an image of the side surface of the subject or the key point position information indicating a position of a body keypoint extracted from the second image and output the angle of the optical axis with respect to a sagittal plane at the time that the second image is captured.

### (Supplementary Note 19)

A posture measurement method for causing a posture measurement apparatus to perform processing of:
holding a shape model representing a shape of a back of a subject as a function of a plurality of parameters;
calculating a value of a predetermined objective function using a candidate point cloud as a candidate of a point cloud indicating the shape of the back of the subject and the shape model as inputs; and
correcting the parameters in such a way as to minimize the value of the predetermined objective function using a constraint condition of the parameters and the shape model as inputs.

### (Supplementary Note 20)

A program causing a computer to execute processing of:
holding a shape model representing a shape of a back of a subject as a function of a plurality of parameters;
calculating a value of a predetermined objective function using a candidate point cloud as a candidate of a point cloud indicating the shape of the back of the subject and the shape model as inputs; and
correcting the parameters in such a way as to minimize the value of the predetermined objective function using a constraint condition of the parameters and the shape model as inputs.

### (Supplementary Note 21)

A posture measurement system including: a posture measurement apparatus and a terminal device capable of communicating with the posture measurement apparatus,
in which the posture measurement apparatus includes:
a holding unit that holds a shape model representing a shape of a back of a subject as a function of a plurality of parameters;
an acquiring unit that acquires, from the terminal device, a candidate point cloud that is a candidate of a point cloud indicating the shape of the back of the subject or an image that is an extraction source of the candidate point cloud;
a calculating unit that calculates a value of a predetermined objective function using the candidate point cloud and the shape model as inputs; and
an optimizing unit that corrects the parameters in such a way as to minimize the value of the predetermined objective function using a constraint condition of the parameters and the shape model as inputs.

Some or all of the elements (for example, configurations and functions) that have been described in Supplementary Notes 2 to 18, which are dependent from Supplementary Note 1, may depend from Supplementary Notes 19, 20 and 21 as well with dependency relationships similar to those of Supplementary Notes 2 to 18. Some or all of the elements that have been described in any supplementary note are applicable to various types of hardware, software, recording means for recording software, systems, and methods.

What is claimed is:

1. A posture measurement apparatus comprising at least one memory configured to store instructions, and
at least one processor configured to execute the instructions to:
hold a shape model representing a shape of a back of a subject as a function of a plurality of parameters;
execute calculation processing of calculating a value of a predetermined objective function using a candidate point cloud as a candidate of a point cloud indicating the shape of the back of the subject and the shape model as inputs; and
execute optimization processing of correcting the parameters in such a way as to minimize the value of the predetermined objective function using a constraint condition of the parameters and the shape model as inputs.

2. The posture measurement apparatus according to claim 1, wherein
the shape model is a model representing a shape of the back of the subject in a sagittal plane, and
the parameters includes at least a size of each part configuring the shape model, an angle representing a posture in a sagittal plane, and position information indicating positions of a neck and a hip joint in the sagittal plane.

3. The posture measurement apparatus according to claim 2, wherein the constraint condition of the parameters includes a condition that the position information as a part of the parameters and candidate position information indicating positions of the neck and the hip joint in the sagittal plane as the candidate point cloud match within a predetermined error range.

4. The posture measurement apparatus according to claim 1, wherein the calculation processing includes processing of calculating, as the value of the predetermined objective function, a sum of distances between each part indicated by the shape model and a closest point in the candidate point cloud.

5. The posture measurement apparatus according to claim 1, wherein the calculation processing includes processing of calculating, as the value of the predetermined objective function, a weighted sum of distances between each part indicated by the shape model and a closest point in the candidate point cloud.

6. The posture measurement apparatus according to claim 1, wherein the calculation processing includes processing of calculating, as the value of the predetermined objective function, a sum of distances between each point of an approximation candidate point cloud obtained by approximating the candidate point cloud based on each part indicated by the shape model and a closest point in the candidate point cloud.

7. The posture measurement apparatus according to claim 1, wherein the calculation processing includes processing of calculating, as the value of the predetermined objective function, a weighted sum of distances between each point of an approximation candidate point cloud obtained by approximating the candidate point cloud based on each part indicated by the shape model and a closest point in the candidate point cloud.

8. The posture measurement apparatus according to claim 1, wherein the at least one processor is configured to execute the instructions to extract the candidate point cloud by inputting a first image obtained by capturing an image of a side surface of the subject in an upright posture or a forward bending posture, determining a region including a back of the subject based on points indicating positions of a neck and a hip joint of the subject designated in the first image, and extracting a point cloud indicating positions of other parts in the region.

9. The posture measurement apparatus according to claim 1, wherein the at least one processor is configured to execute the instructions to extract the candidate point cloud by inputting a first image obtained by capturing an image of a side surface of the subject in an upright posture or a backward bending posture, detecting a back surface edge line indicating an edge of a back surface of the subject and a front surface edge line indicating an edge of a front surface in the first image, and translating a center line between the back surface edge line and the front surface edge line or the front surface edge line toward the back surface edge line in such a way as to match a part of the back surface edge line.

10. The posture measurement apparatus according to claim 1, wherein the at least one processor is configured to execute the instructions to input the parameters corrected in the optimization processing and the candidate point cloud, and determine both end points of a curve as both end points of the candidate point cloud based on positions of parts configuring the corrected shape model in such a way as to extract only a designated range of the curve connecting the points of the candidate point cloud.

11. The posture measurement apparatus according to claim 1, wherein the at least one processor is configured to execute the instructions to obtain a point cloud representing the shape of the back of the subject by inputting the parameters corrected in the optimization processing and the candidate point cloud, and obtaining a weighted average by adding a predetermined weight to each point of the point cloud indicating the position of each part configuring the shape model indicated by the corrected parameters and the candidate point of the candidate point cloud associated to each point.

12. The posture measurement apparatus according to claim 1, wherein the at least one processor is configured to execute the instructions to extract key point position information from a second image by using a learned model machine-learned in such a way as to input the second image obtained by capturing an image of a side surface of the subject and output the key point position information indicating a position of a body keypoint.

13. The posture measurement apparatus according to claim 1, wherein the at least one processor is configured to execute the instructions to extract a feature regarding a state of each part of the subject indicated by the shape of the back of the subject based on a point cloud indicating a position of each part configuring the shape model indicated by the parameters corrected in the optimization processing.

14. The posture measurement apparatus according to claim 13, wherein the at least one processor is configured to execute the instructions to estimate a state of each part of the subject based on the extracted feature.

15. The posture measurement apparatus according to claim 1, wherein
the at least one processor is configured to execute the instructions to receive an input of key point position information indicating a position of a body keypoint based on a user operation, and
the holding the shape model includes determining the parameters to be held based on the input key point position information.

16. The posture measurement apparatus according to claim 1, wherein the at least one processor is configured to execute the instructions to receive an input of the candidate point cloud group based on a user operation.

17. The posture measurement apparatus according to claim 1, wherein the at least one processor is configured to execute the instructions to display, on a display device, a point cloud indicating a position of each part configuring the shape model indicated by the parameters corrected in the optimization processing.

18. The posture measurement apparatus according to claim 1, wherein the at least one processor is configured to execute the instructions to estimate an angle of an optical axis from a second image or key point position information by using a learned model machine-learned in such a way as to input the second image obtained by capturing an image of the side surface of the subject or the key point position information indicating a position of a body keypoint extracted from the second image and output the angle of the optical axis with respect to a sagittal plane at the time that the second image is captured.

19. A posture measurement method for causing a posture measurement apparatus to perform processing of:
holding a shape model representing a shape of a back of a subject as a function of a plurality of parameters;
calculating a value of a predetermined objective function using a candidate point cloud as a candidate of a point cloud indicating the shape of the back of the subject and the shape model as inputs; and
correcting the parameters in such a way as to minimize the value of the predetermined objective function using a constraint condition of the parameters and the shape model as inputs.

20. A non-transitory computer readable medium storing a program for causing a computer to execute the following processing of:
holding a shape model representing a shape of a back of a subject as a function of a plurality of parameters;
calculating a value of a predetermined objective function using a candidate point cloud as a candidate of a point cloud indicating the shape of the back of the subject and the shape model as inputs; and
correcting the parameters in such a way as to minimize the value of the predetermined objective function using a constraint condition of the parameters and the shape model as inputs.
